(19) United States
(12) Patent Application Publication
(10) Pub. No.: US 2024/0307702 A1
(43) Pub. Date: Sep. 19, 2024

(54) METHOD AND DEVICE FOR DELIVERING MULTI-PHASE DEFIBRILLATION THERAPY

(71) Applicant: Pacesetter, Inc., Sylmar, CA (US)

(72) Inventors: Reza Shahandeh, Los Angeles, CA (US); Wenwen Li, San Jose, CA (US); Gene Bornzin, Sylmar, CA (US); Gabriel Mouchawar, Sylmar, CA (US); Alan Vogel, Sylmar, CA (US); Kyungmoo Ryu, Sylmar, CA (US)

(21) Appl. No.: 18/675,446

(22) Filed: May 28, 2024

Related U.S. Application Data
(62) Division of application No. 16/722,195, filed on Dec. 20, 2019.
(60) Provisional application No. 62/786,636, filed on Dec. 31, 2018.

(51) Int. Cl. A61N 1/39

(52) U.S. Cl. CPC A61N 1/3956; A61N 1/3981

(57) ABSTRACT

Methods and systems are provided that comprise: sensing cardiac events of a heart; utilizing one or more processors to perform: declaring a ventricular fibrillation (VF) episode based on the cardiac events charging a single charge storage capacitor; delivering a multi-phase VF therapy that includes phase I and phase II therapies, wherein: a) during the phase I therapy, a combination of two or more medium voltage (MV) shocks are delivered entirely from the single charge storage capacitor; and b) during the phase II therapy, a low voltage pulse train is delivered at least partially from the single charge storage capacitor. Methods and systems are provided that comprise delivering first and second pulses of at least a first biphasic shock, wherein a parallel-series reconfiguration circuit connects and configures the capacitors of the capacitor bank in a parallel configuration to deliver a parallel biphasic shock; connecting the capacitors of the capacitor bank in a series configuration; and delivering first and second pulses of a second biphasic shock while the capacitors are connected in series to deliver a series biphasic shock.

### REFERENCE TO RELATED APPLICATIONS

The present application is a divisional of U.S. application Ser. No. 16/722,195, filed 20 Dec. 2019, which claims priority to U.S. Application No. 62/786,636, filed 31 Dec. 2018, the complete subject matter of which are expressly incorporated herein by reference in their entirety.

### BACKGROUND

Embodiments of the present disclosure generally relate to methods and devices for treating ventricular fibrillation (VF) arrhythmias and more particularly to methods and devices that deliver VF therapy in a manner that reduces a defibrillation threshold (DFT) needed to convert the VF arrhythmia.

High voltage ventricular tachy therapies are delivered by an implantable cardioverter defibrillator (ICD) devices and cardiac resynchronization therapy defibrillator (CRT-D) devices after the tachycardia episode is detected and classified. Currently marketed ICD and CRT-D devices use a conventional bi-phasic capacitive discharge waveform that is delivered from a bank of multiple capacitors that are connected in series. Conventional ICD and CRT-D devices deliver between 35 and 42 joules of energy in a single bi-phasic shock. In order to generate a high energy shock of 40 J or more, conventional ICD and CRT-D devices require a bank of two or more high voltage capacitors connected in series and typically charged to 800V-900V. The capacitor bank and battery are two of the larger components in ICD and CRT-D devices and thus the overall size of the device is largely dependent on the space needed to house the capacitor bank and battery. For example, the space requirements of the capacitor bank and battery cause the ICD and CRT-D devices to be 29 cc or larger.

The capacitor bank, when charged to 800V-900V, delivers a high voltage waveform that has a leading edge that starts at or near 800V-900V. The high voltage leading edge of the shock waveform has been recognized as a possible source of damage to the heart tissue near the electrodes that delivery the shock.

A need remains for methods and devices that produce an effective defibrillation therapy at a significantly reduced voltage level that reduces a risk of tissue damage at the point of therapy delivery and that enable a significant size reduction in the implantable device.

### BRIEF SUMMARY

In accordance with embodiments herein, a method is provided that comprises: sensing cardiac events of a heart; utilizing one or more processors to perform: declaring a ventricular fibrillation (VF) episode based on the cardiac events; charging a single charge storage capacitor; delivering a multi-phase VF therapy that includes phase I and phase II therapies, wherein: a) during the phase I therapy, a combination of two or more medium voltage (MV) shocks are delivered entirely from the single charge storage capacitor; and b) during the phase II therapy, a low voltage pulse train is delivered at least partially from the single charge storage capacitor.

In accordance with embodiments herein, the multi-phase VF therapy further includes a phase III therapy and the delivering further comprises c) delivering pacing pulses during the phase III therapy. In accordance with embodiments herein, an entirety of the phase I therapy is delivered from the single charge storage capacitor based on a single charge of the single charge storage capacitor. In accordance with embodiments herein, the MV shock represents at least one of: i) a defibrillation stimulus delivered at a select energy level that is at least one of i) no more than 25 J or ii) has a maximum voltage of no more than 500V; or ii) a defibrillation stimulus delivered at a select energy level that is at least one of i) between 15 J-25 J or ii) has a maximum voltage of between 100V-475V. In accordance with embodiments herein, the method may further comprise charging the single capacitor to a full voltage of between 400 V-475 V; during the phase I therapy, delivering the combination of two or more MV shocks at first and second voltage levels, respectively, below the full voltage, such that the single capacitor retains a post phase I voltage following an end of the phase I therapy; and delivering the low voltage pulse train at a low-voltage below the post phase I voltage. In accordance with embodiments herein, the method may further comprise: charging the single capacitor to a full charge of no more than 25 J; managing an energy discharge from the single capacitor during the phase I therapy to retain a select post-phase I residual charge; and during the phase II therapy delivering the low voltage pulse train utilizing the post-phase I residual charge, followed by pacing pulses.

In accordance with embodiments herein, an implantable medical device is provided that comprises: electrodes configured to sense cardiac events; a battery and a single charge storage capacitor; one or more processors configured to: declare a ventricular fibrillation (VF) episode based on the cardiac events; charge a single charge storage capacitor utilizing the battery; deliver a multi-phase VF therapy that includes phase I and II therapies, wherein: a) during the phase I therapy, a combination of two or more medium voltage (MV) shocks are delivered entirely from the single charge storage capacitor; and b) during the phase II therapy, a low voltage pulse train is delivered at least partially from the single charge storage capacitor.

In accordance with embodiments herein, the one or more processors are further configured to deliver, as part of the multi-phase VF therapy, a phase III therapy that includes pacing pulses. In accordance with embodiments herein, an entirety of the phase I is delivered from the single charge storage capacitor based on a single charge of the single charge storage capacitor. In accordance with embodiments herein, the MV shock represents at least one of: i) a defibrillation stimulus delivered at a select energy level that is at least one of i) no more than 25 J or ii) has a maximum voltage of no more than 500V; or ii) a defibrillation stimulus delivered at a select energy level that is at least one of i) between 15 J-25 J or ii) has a maximum voltage of between 100V-475V. In accordance with embodiments herein, the one or more processors are further configured to: charge the single capacitor to a full voltage of between 400 V-475 V; during the phase I therapy, deliver the combination of two or more MV shocks at first and second voltage levels, respectively, below the full voltage, such that the single capacitor retains a post phase I voltage following an end of the phase I therapy; and deliver the low voltage pulse train at a low-voltage below the post phase I voltage. In accordance with embodiments herein, the one or more processors are further configured to: charge the single capacitor to a full charge of no more than 25 J; manage an energy discharge from the single capacitor during the phase I therapy to retain a select post-phase I residual charge; and during the phase II therapy, deliver the low voltage pulse train utilizing the post-phase I residual charge, followed by pacing pulses. In accordance with embodiments herein, the one or more processors are further configured to, after delivery of the phase I therapy and before delivery of the phase II therapy: sense additional cardiac events; and when the additional cardiac events indicate that the VF episode has been terminated by the phase I therapy, suspend or cancel the phase II therapy before delivering the phase II therapy.

In accordance with the embodiment herein, an output control system for use in an implantable medical device is provided, comprising: an output configured to be connected to a lead; a charging circuit; a capacitor switchably coupled to the charging circuit; a switching circuit coupled between the capacitor and the output; an output control circuit configured to generate a control signal that includes control pulses that control the switching circuit to switchably connect the capacitor to the output, the output control circuit configured to vary a duty cycle of the control pulses in a manner that defines a shape of an effective waveform for one or more shocks.

In accordance with embodiments herein, the shape of the effective waveform includes a positive phase segment that is defined by a collection of the control pulses, in which the duty cycle varies such that an initial portion of the collection includes a first duty cycle and a final portion of the collection includes a second duty cycle that is longer than the first duty cycle. In accordance with embodiments herein, the output control circuit is configured to vary the first and second duty cycles to define a leading edge profile having an ascending non-linear shape that transitions from a low voltage to a high voltage, and a steady-state profile at the high voltage for a duration of the positive phase segment. In accordance with embodiments herein, the output control circuit is configured to increase the duty cycle of the successive control pulses from an initial shorter duty cycle to a final longer duty cycle. In accordance with embodiments herein, the capacitor is configured to store a full charge with a voltage of at least 400V, and the output control circuit is configured to vary the duty cycle of the control pulses to define the effective waveform of a first shock to have a voltage of less than 300V. In accordance with embodiments herein, the capacitor represents a single charge storage capacitor that is configured to store a full charge with a voltage of no more than 500V, and the output control circuit is configured to vary the duty cycle of the control pulses to define the effective waveform for first and second shocks to be delivered from the single charge storage capacitor based on a single charge of the single charge storage capacitor. In accordance with embodiments herein, the capacitor represents a single charge storage capacitor that is configured to store a full charge with a voltage of no more than 500V, and the output control circuit is configured to vary the duty cycle of the control pulses to deliver a multi-phase VF therapy that includes first and second phase therapies, wherein: a) during the first phase therapy, a combination of two or more medium voltage (MV) shocks are delivered entirely from the single charge storage capacitor; and b) during the second phase therapy, a low voltage pulse train is delivered at least partially from the single charge storage capacitor.

In accordance with embodiments herein, an implantable medical device is provided that comprises: electrodes configured to sense cardiac events; a charging circuit; a capacitor switchably coupled to the charging circuit; a switching circuit coupled between the capacitor and the output; an output control circuit configured to generate a control signal that includes control pulses that controls the switching circuit to switchably connect the capacitor to the electrodes, the output control circuit configured to vary a duty cycle of the control pulses in a manner that defines a shape of an effective waveform for one or more shocks.

In accordance with embodiments herein, the shape of the effective waveform includes a positive phase segment that is defined by a collection of the control pulses, in which the duty cycle varies such that an initial portion of the collection includes a first duty cycle and a final portion of the collection includes a second duty cycle that is longer than the first duty cycle. In accordance with embodiments herein, the output control circuit is configured to linearly increase the duty cycle of the successive control pulses from an initial shorter duty cycle to a final longer duty cycle. In accordance with embodiments herein, the capacitor is configured to store a full charge with a voltage of at least 400V, and the output control circuit is configured to vary the duty cycle of the control pulses to define the effective waveform of a first shock to have a voltage of less than 300V. In accordance with embodiments herein, the capacitor represents a single charge storage capacitor that is configured to store a full charge with a voltage of no more than 500V, and the output control circuit is configured to vary the duty cycle of the control pulses to define the effective waveform for first and second shocks to be delivered from the single charge storage capacitor based on a single charge of the single charge storage capacitor. In accordance with embodiments herein, the capacitor represents a single charge storage capacitor that is configured to store a full charge with a voltage of no more than 500V, and the output control circuit is configured to vary the duty cycle of the control pulses to deliver a multi-phase VF therapy that includes first and second phase therapies, wherein: a) during the first phase therapy, a combination of two or more medium voltage (MV) shocks are delivered entirely from the single charge storage capacitor; and b) during the second phase therapy, a low voltage pulse train is delivered at least partially from the single charge storage capacitor. In accordance with embodiments herein, the output control circuit is configured to vary the first and second duty cycles to define a leading edge profile having an ascending non-linear shape that transitions from a low voltage to a high voltage, and a steady-state profile at the high voltage for a duration of the positive phase segment.

In accordance with embodiments herein, the method is provided that comprises: sensing cardiac events of a heart; utilizing one or more processors to declare a ventricular fibrillation (VF) episode based on the cardiac events and initiate charge of a capacitor based on the declaration of the VF episode; switchably connect the capacitor to the output based on a control signal; generate the control signal to includes control pulses having a duty cycle; varying the duty cycle of the control pulses in a manner that defines a shape of an effective waveform for one or more shocks.

In accordance with embodiments herein, the method further comprises utilizing a collection of the control pulses to define the shape of the effective waveform to include a positive phase segment, in which the duty cycle varies such that an initial portion of the collection includes a first duty cycle and a final portion of the collection includes a second duty cycle that is longer than the first duty cycle. In accordance with embodiments herein, the method further comprises varying the first and second duty cycles to define a leading edge profile having an ascending non-linear shape that transitions from a low voltage to a high voltage, and a steady-state profile at the high voltage for a duration of the positive phase segment. In accordance with embodiments herein, the method further comprises storing a full charge on the capacitor with a voltage of at least 400V, and varying the duty cycle of the control pulses to define the effective waveform of a first shock to have a voltage of less than 300V. In accordance with embodiments herein, the capacitor represents a single charge storage capacitor that is configured to store a full charge with a voltage of no more than 500V, and the output control circuit is configured to vary the duty cycle of the control pulses to define the effective waveform for first and second shocks to be delivered from the single charge storage capacitor based on a single charge of the single charge storage capacitor. In accordance with embodiments herein, the method further comprises storing a full charge of the capacitor in a single charge storage capacitor with a voltage of no more than 500V, and varying the duty cycle of the control pulses to deliver a multi-phase VF therapy that includes first and second phase therapies, wherein: a) during the first phase therapy, a combination of two or more medium voltage (MV) shocks are delivered entirely from the single charge storage capacitor; and b) during the second phase therapy, a low voltage pulse train is delivered at least partially from the single charge storage capacitor. In accordance with embodiments herein, the method further comprises increasing the duty cycle of the successive control pulses from an initial shorter duty cycle to a final longer duty cycle.

In accordance with embodiments herein, increasing the effective capacitance that is utilized in connection with delivering one or more MV shocks will reduce the voltage requirement for the initial charge of the capacitor bank. As one nonlimiting example, utilizing an effective capacitance of approximate 440 μF can reduce the voltage requirement by approximate 35%. As another example, delivering an MV shock having a waveform defined by an effective capacitance of approximately 440 μF and an initial maximum voltage of 490 V is substantially equivalent to delivering an HV shock having a waveform defined by an effective capacitance of approximately 110 μF and an initial maximum voltage of 753 V.

In accordance with embodiments herein, a method comprises delivering first and second pulses of at least a first biphasic shock, wherein a parallel-series reconfiguration circuit connects and configures the capacitors of the capacitor bank in a parallel configuration to deliver a parallel biphasic shock; connecting the capacitors of the capacitor bank in a series configuration; and delivering first and second pulses of a second biphasic shock while the capacitors are connected in series to deliver a series biphasic shock. Optionally, the capacitors may have a capacitance of between 200 μF and 350 μF and be charged to 450V-500V. Optionally, when connected in parallel the effective capacitance of the capacitor bank is between 400 and 500 μF, and more preferably 440 μF, with a maximum leading voltage of 450V-500V. When connected in series, the effective capacitance is between 100 μF and 200 μF, and more preferably 110 μF with a maximum leading voltage of the sum of the voltage on each capacitor. In accordance with embodiments herein, the PSR circuit reconfigures the capacitor bank to have first and second effective capacitances during first and second biphasic shocks, respectively. The first and second biphasic shocks similarly exhibit first and second exponential decays, respectively.

In accordance with embodiments herein, the method may further comprise delivering a third biphasic shock, before the second biphasic shock, with the capacitors of the capacitor bank in the parallel configuration, the third biphasic shock with the parallel configuration being delivered after a predetermined shock to shock interval based on a cycle length of the VF episode. Optionally, the first and third biphasic shocks delivered with the parallel configuration are delivered without recharging the capacitor bank between the first and third biphasic shocks. Optionally, the PSR circuit 908 may maintain the parallel configuration for more than one biphasic shock, such as for a series of MV shocks as described above in connection with various phase I therapies.

In accordance with embodiments herein, an implantable medical device is provided that comprises: electrodes configured to sense cardiac events; a charging circuit; a reconfigurable capacitor bank that includes capacitors; a switching circuit coupled between the reconfigurable capacitor bank and the output; and a parallel/series reconfiguration (PSR) circuit that interconnects the capacitors in parallel and series configurations, the PSR circuit configured to switch between the parallel and series configurations during delivery of a shock to define an ascending stepped waveform.

In accordance with embodiments herein, the shape of the ascending stepped waveform includes a positive phase segment that includes first and second waveform segments that are defined by the parallel and series configurations, respectively, of the capacitors. Optionally, the ascending stepped waveform represents a biphasic waveform, the PSR circuit configured to connect the capacitors in the parallel configuration during the discharge of a first portion of a positive phase of the biphasic waveform, the PSR circuit configured to switch the capacitors from the parallel configuration to the series configuration during the discharge of a second portion of the positive phase of the biphasic waveform. Optionally, the PSR circuit is configured to switch the capacitors from the series configuration back to the parallel configuration during the discharge of a negative phase of the biphasic waveform. Optionally, the PSR circuit is configured to switch the capacitors from the parallel configuration, to the series configuration and back to the parallel configuration at intermediate points during discharge of the shock. Optionally, the reconfigurable capacitor bank includes first and second capacitors, each of which has an individual capacitance value of between 55 μF and 220 μF, the PSR circuit switching an effective capacitance of the reconfigurable capacitor bank between 27.5 μF and 440 μF when switching between the series configuration and the parallel configuration, respectively. Optionally, an output control circuit is provided that is configured to generate a control signal that includes control pulses that control the switching circuit to switchably connect the configurable capacitor bank to the electrodes, the output control circuit configured to vary a duty cycle of the control pulses in a manner that defines a shape of one or more of first, second or third waveform segments of the shock.

In accordance with embodiments herein, the method is provided that comprises: sensing cardiac events of a heart; utilizing one or more processors to declare a ventricular fibrillation (VF) episode based on the cardiac events and initiate charge of a reconfigurable capacitor bank based on the declaration of the VF episode; switchably connect capacitor of the reconfigurable capacitor bank to the output to deliver a shock; and during delivery of the shock, connecting the capacitors in a parallel configuration and switching from the parallel configuration to a series configuration to define an ascending stepped waveform for the shock.

In accordance with embodiments herein, the method shapes the ascending stepped waveform to include a positive phase segment that includes first and second waveform segments that are defined the parallel and series configurations, respectively, of the capacitors. Optionally, the ascending stepped waveform represents a biphasic waveform, the connecting further including connecting the capacitors in the parallel configuration during the discharge of a first portion of a positive phase of the biphasic waveform, and switching the capacitors from the parallel configuration to the series configuration during the discharge of a second portion of the positive phase of the biphasic waveform. Optionally, the method further comprises switching the capacitors from the series configuration back to the parallel configuration during the discharge of a negative phase of the biphasic waveform. Optionally, the connecting comprises switching the capacitors from the parallel configuration, to the series configuration and back to the parallel configuration at intermediate points during the discharge of the shock. Optionally, the method further comprises generating a control signal that includes control pulses that control a switching circuit to switchably connect the configurable capacitor bank to the electrodes, the circuit signal varying a duty cycle of the control pulses in a manner that defines a shape of one or more of first, second or third waveform segments of the shock.

### BRIEF DESCRIPTION OF THE DRAWINGS

FIG. 1 illustrates an IMD and external device coupled to a heart in a patient and implemented in accordance with embodiments herein.

FIG. 2A illustrates a block diagram of an exemplary IMD that is configured to be implanted into the patient in accordance with embodiments herein.

FIG. 2B illustrates a graphical representation of an implantable medical system that is configured to apply multi-phase VF therapy in accordance with embodiments herein.

FIG. 3 illustrates a process for coordinating a multi-phase VF therapy that includes phase I, II, III therapies in accordance with embodiments herein.

FIG. 4 illustrates example waveforms that may be delivered during the phase I and phase II therapies in accordance with embodiments herein.

FIG. 5 illustrates example waveforms that may be delivered during the phase I and phase II therapies in accordance with embodiments herein.

FIG. 6 illustrates a block diagram of an output control circuit formed in accordance with embodiments herein.

FIG. 7 illustrates an example of a control signal to manage the switches within the control circuit of FIG. 6 in accordance with embodiments herein.

FIG. 8 illustrates an example of an effective stimulation waveform experienced by a heart during delivery of a single biphasic MV shock in accordance with embodiments herein.

FIG. 9A illustrates a schematic diagram of at least a portion of a circuit utilized in an IMD to manage delivery of one or more biphasic shocks in accordance with embodiments herein.

FIG. 9B illustrates a flowchart that may be implemented in connection with embodiments herein to deliver more than one shock while switching between capacitor series and parallel configurations in accordance with embodiments herein.

FIG. 9C illustrates a flowchart that may be implemented in connection with embodiments herein to deliver more than one shock while switching between capacitor series and parallel configurations in accordance with embodiments herein.

FIG. 10 illustrates a theoretical response of the cell membrane voltage when a unitary biphasic defibrillation shock is delivered from a capacitor bank exhibiting a 110 μF effective capacitance in accordance with embodiments herein.

FIG. 11 shows the response to the same amplitude pulse as in FIG. 10 but derived from a 440 μF effective capacitance in accordance with embodiments herein.

FIG. 12A Illustrates a series of theoretical responses for membrane potentials that are calculated from convolutions of corresponding defibrillation forcing functions and a predetermined consistent membrane impulse response in accordance with embodiments herein.

FIG. 12B presents an example model of an ascending stepped waveform formed along with a model of the membrane response thereto in accordance with embodiments herein.

FIG. 12C illustrates a collection of ascending stepped waveforms and corresponding membrane responses that were modeled utilizing the various shocking load resistances in accordance with embodiments herein.

FIG. 12D illustrates predicted delivered energy as a function of the shocking load resistances in accordance with embodiments herein.

FIG. 12E illustrates predicted peak voltage as a function of the shocking load resistances in accordance with embodiments herein.

FIG. 12F illustrates the energy predictions of FIG. 12D normalized as a function of shocking load resistance in accordance with embodiments herein.

FIG. 12G illustrates the voltage predictions of FIG. 12E normalized as a function of shocking load resistance in accordance with embodiments herein.

FIG. 12H illustrates graphs plotting examples of pulse widths that may be utilized in connection with the first, second and third waveform segments of the ascending stepped waveform to achieve a desired membrane response in accordance with embodiments herein.

FIG. 12I illustrates graphs plotting examples of pulse widths that may be utilized in connection with the first, second and third waveform segments of the ascending stepped waveform to achieve a desired membrane response in accordance with embodiments herein.

FIG. 12J illustrates graphs plotting examples of pulse widths that may be utilized in connection with the first, second and third waveform segments of the ascending stepped waveform to achieve a desired membrane response in accordance with embodiments herein.

FIG. 12K illustrates graphs plotting examples of pulse widths that may be utilized in connection with the first and second phases of a conventional truncated exponential biphasic waveform the conventional waveform includes a first phase and a second phase in accordance with embodiments herein.

FIG. 13 illustrates a graph plotting different effective capacitances (along the horizontal axis) and normalized DFT voltages (along the vertical axis).

FIG. 14 illustrates an electrical model for a myocardial cellular membrane.

FIG. 15 illustrates a series of equations to model the membrane impulse response in accordance with embodiments herein.

FIG. 16 illustrates a series of equations to model the defibrillation forcing function in accordance with embodiments herein.

FIG. 17 illustrates an equation to model the membrane voltage/potential experienced by the myocardial tissue when driven by a defibrillation shock in accordance with embodiments herein.

FIG. 18 illustrates equations to calculate pulse widths for the pulses of one or more of the shocks in the phase I therapy, the pulse train in the phase II therapy, and/or the pacing pulses in the phase III therapy.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods and systems described herein may employ all or portions of structures or aspects of various embodiments discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, where indicated, the methods may be fully or partially implemented by one or more processors of one or more devices or systems. While the operations of some methods may be described as performed by the processor(s) of one device, additionally, some or all of such operations may be performed by the processor(s) of another device described herein.

### Definitions

The term “obtain” or “obtaining”, as used in connection with data, signals, information and the like, includes at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The terms “multi-phase defibrillation threshold” or “multi-phase DFT” refer to a minimum amount of energy needed to be delivered in an MV shock, in combination with phase II and/or phase III therapies, in order to return a heart to a normal rhythm from a condition in which the heart is experiencing a fibrillation dysrhythmia episode.

The terms “shock-only defibrillation threshold” or “shock-only DFT” refer to a minimum amount of energy needed to be delivered in an HV shock, alone without any phase II and/or phase III therapy, in order to return a heart to a normal rhythm from a condition in which the heart is experiencing a fibrillation dysrhythmia episode.

The terms “medium-voltage shock” and “MV shock” refer to defibrillation stimulus delivered at an energy level sufficient to terminate a defibrillation episode in a heart, wherein the energy level is defined in Joules, pulse width, and/or maximum charge voltage. A MV shock from an IMD with a transvenous lead will have a different maximum energy and/or charge voltage than an MV shock from a subcutaneous IMD with a subcutaneous lead. In connection with an IMD having a transvenous lead, the terms medium voltage shock and MV shock refer to defibrillation stimulation that has an energy level that is no more than 25 J, and more preferably 15 J-25 J and/or has a maximum voltage of no more than 500V, preferably between 100-475V and more preferably between 400V-475V. In connection with an IMD having a subcutaneous lead (e.g., parasternal or otherwise), the terms medium voltage shock and MV shock refer to defibrillation stimulation that has an energy level that is no more than 48 J, and more preferably 35 J-45 J and/or has a maximum voltage of no more than 750 V, preferably between 200V-750V and more preferably between 500V-750V.

The terms “low-voltage pulse train”, “LV pulse train”, “low voltage shock”, “low voltage stimulation”, “LV shock” and the like, refer to stimulus delivered at an energy level below an MV shock energy level, and above a pacing pulse energy level, wherein the energy level is defined in Joules, maximum charge voltage and/or pulse width. In connection with an IMD having a transvenous lead, the foregoing terms refer to stimulation that has an energy level below the energy of a MV shock and a pulse width of between 5 ms and 15 ms, and more preferably 7 ms to 10 ms and even more preferably about 10 ms.

The terms “high-voltage shock” and “HV shock” refer to defibrillation stimulus delivered at an energy level sufficient to terminate a defibrillation episode in a heart. In connection with an IMD having a transvenous lead, the energy level is defined in Joules to be 40 J or more and/or the energy level is defined in terms of voltage to be 750V or more. In connection with an IMD having a subcutaneous lead (e.g., parasternal or otherwise), the terms high voltage shock and HV shock refer to defibrillation stimulation that has an energy level that 65 J or more, and more preferably 80 J and/or has a maximum voltage of more than 750 V, and more preferably a voltage of 850V-1000V.

The terms “high frequency” and “HF”, as used in connection with pacing pulses and pacing therapy refer to delivering pacing pulses at a rate greater than a rate associated with anti-tachycardia pacing, namely at a rate of at least 30 Hz.

The terms “single charge storage capacitor”, “single capacitor”, and any similar terms used in connection with describing a “single” and a “capacitor”, shall mean that a single physical component is utilized to perform the corresponding operation (e.g., retain a charge, delivery a shock, maintain a defined initial or later voltage), and shall expressly exclude the use of more than one physical component. By way of example, the terms “single charge storage capacitor”, “single capacitor”, and any similar terms used in connection with describing a “single” and a “capacitor”, shall expressly exclude configurations in which two or more physical capacitors are coupled in parallel or series with one another, or utilized independently to delivery corresponding portions of a shock.

### Overview

Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. For example, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference. Additionally or alternatively, the IMD may include one or more structural and/or functional aspects of the device(s) described in U.S. Pat. No. 9,216,285 “Leadless Implantable Medical Device Having Removable and Fixed Components” and U.S. Pat. No. 8,831,747 “Leadless Neurostimulation Device and Method Including the Same”, which are hereby incorporated by reference. Additionally or alternatively, the IMD may include one or more structural and/or functional aspects of the device(s) described in U.S. Pat. No. 8,391,980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the IMD may be a subcutaneous IMD that includes one or more structural and/or functional aspects of the device(s) described in U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

Embodiments herein provide methods and systems that generate a new and unique multi-phase shock waveform that has a multi-phase DFT that is lower than a shock-only DFT. The multi-phase shock waveform includes three phases. The phase I therapy includes a combination of two or more medium voltage shocks delivered within one episode cycle length (CL). The phase II therapy includes a low voltage pulse train (e.g., a collection of 4 or more low voltage pulses) delivered at slightly higher voltage than a ventricular capture threshold, where the pulse train is delivered within an interval shorter than one CL. The phase III therapy includes a burst of pacing pulses delivered at potentially higher rates or appropriately timed with respect to a sensed intrinsic event. The sequence of multiple shocks (at medium and low voltages), followed by burst pacing lowers the total energy required to terminate VF, as compared to the total energy delivered in a single high voltage shock.

Ventricular fibrillation (VF) is caused by a multi-reentrant electrical loop in the heart. In contrast, Ventricular tachycardia (VT) is caused by a single electrical loop. Treating VT is therefore simpler using ATP alone and potentially with a lower voltage shock if ATP is not successful. VF on the other hand cannot be terminated with ATP due to its nature of being a more complex problem. In order to reduce the shock voltage required to terminate VF, embodiments herein use a novel shock sequence which could break multiple re-entrant circuits that sustain VF. Embodiments herein also describe the designs of implantable cardioverter defibrillator or a subcutaneous IMD that utilizes unique control circuits to manage the waveform shapes of the MV and/or LV shocks.

While various embodiments herein are described in connection with new and unique multi-phase shock waveforms, in accordance with at least some embodiments a single phase shock waveform may be delivered with the single phase shock waveform including more than one shock pulse.

### Implantable Medical Device

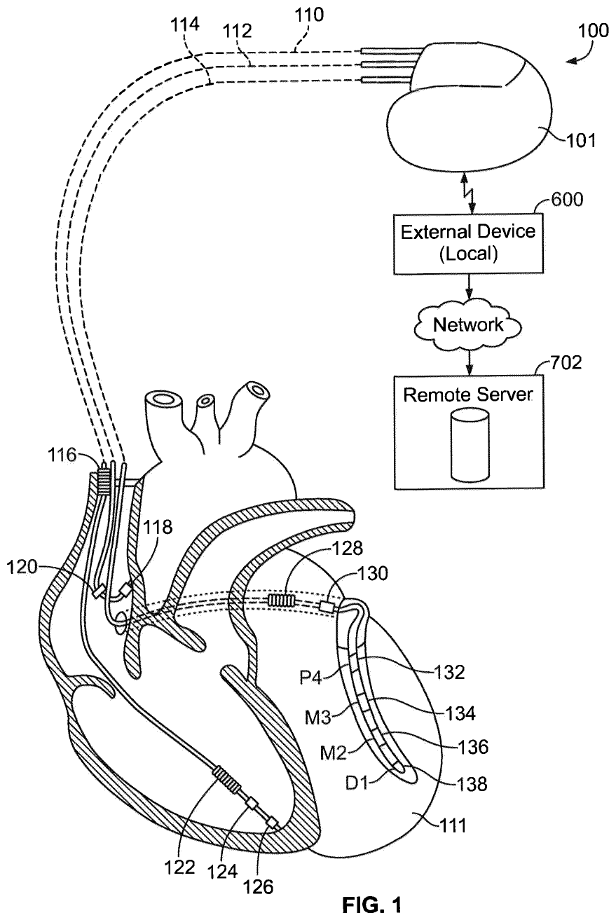

FIG. 1 illustrates an IMD 100 and external device 600 coupled to a heart 111 in a patient and implemented in accordance with one embodiment. The external device 600 may be a programmer, an external defibrillator, a workstation, a portable computer, a personal digital assistant, a cell phone, a bedside monitor and the like. The IMD may represent a cardioverter, cardiac rhythm management device, defibrillator and the like, implemented in accordance with embodiments herein. The IMD 100 may be a dual-chamber stimulation device capable of treating both fast and slow arrhythmias with stimulation therapy, including cardioversion, defibrillation, anti-tachycardia pacing and pacing stimulation, as well as capable of detecting heart failure, evaluating its severity, tracking the progression thereof, and controlling the delivery of therapy and warnings in response thereto. Exemplary structures for the IMD 100 are discussed and illustrated in the drawings herewith.

The IMD 100 communicates with a local external device. The local external device communicates with a remote server. The IMD 100 includes a housing 101 that is joined to a header assembly that holds receptacle connectors connected to a right ventricular lead 110, a right atrial lead 112, and a coronary sinus lead 114, respectively. The leads 112, 114 and 110 measure cardiac signals of the heart 111. The right atrial lead 112 includes an atrial tip electrode 118 and an atrial ring electrode 120. The coronary sinus lead 114 includes a left atrial ring electrode 128, a left atrial coil electrode 130 and one or more left ventricular electrodes 132-138 (e.g., also referred to as P1, M1, M2 and D1) to form a multi-pole LV electrode combination. The right ventricular lead 110 includes an RV tip electrode 126, an RV ring electrode 124, an RV coil electrode 122, and an SVC coil electrode 116. The leads 112, 114 and 110 detect IEGM signals that are processed and analyzed as described herein. The leads 112, 114 and 110 also delivery therapies as described herein.

During implantation, the external device 600 is connected to one or more of the leads 112, 114 and 110 through temporary inputs. The inputs of the external device 600 receive IEGM signals from the leads 112, 114 and 110 during implantation and display the IEGM signals to the physician on a display. Optionally, the external device 600 may not be directly connected to the leads 112, 114 and 110. Instead, the IEGM cardiac signals sensed by the leads 112, 114 and 110 may be collected by the IMD 100 and then transmitted wirelessly to the external device 600. Hence, the external device 600 receives the IEGM cardiac signals through telemetry circuit inputs. The physician or another user controls operation of the external device 600 through a user interface.

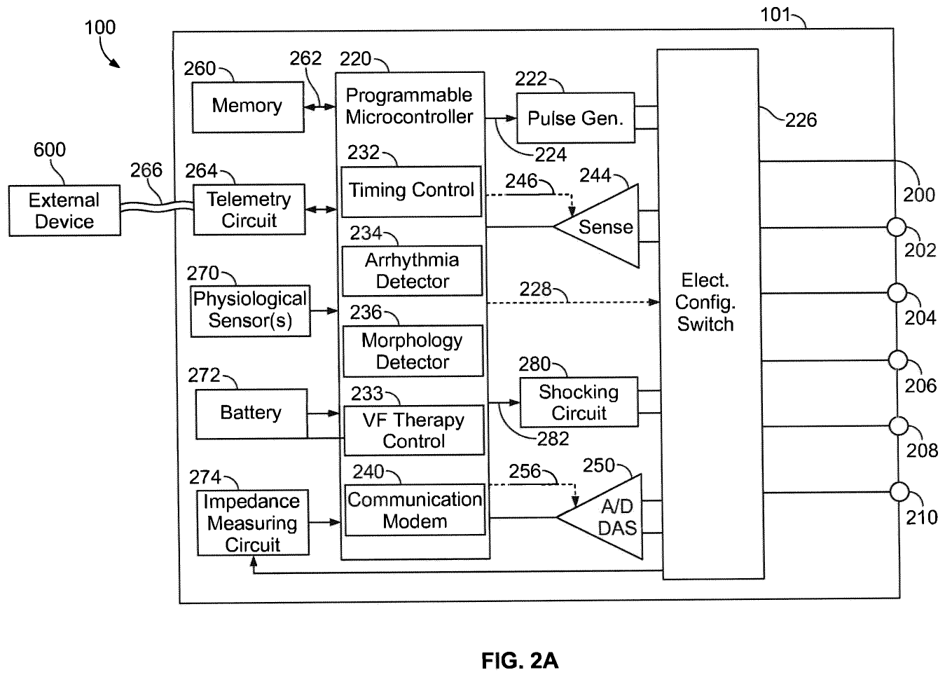

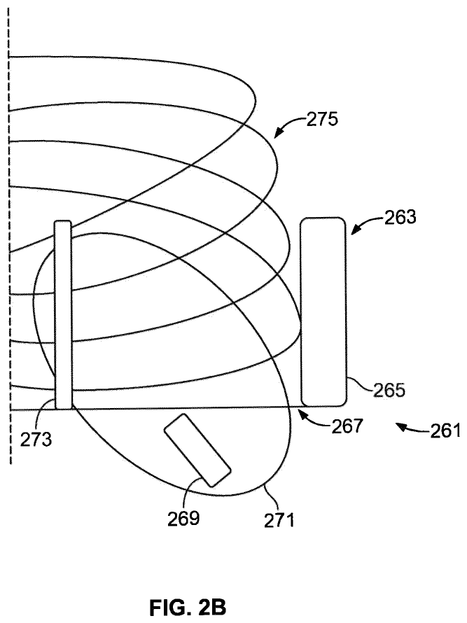

FIG. 2A shows a block diagram of an exemplary IMD 100 that is configured to be implanted into the patient. The IMD 100 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 100 has a housing 101 to hold the electronic/computing components. The housing 101 (which is often referred to as the “can,” “case,” “encasing,” or “case electrode”) may be programmably selected to act as the return electrode for certain stimulus modes. The housing 101 further includes a connector (not shown) with a plurality of terminals 200-210. The terminals may be connected to electrodes that are located in various locations within and about the heart. The type and location of each electrode may vary. For example, the electrodes may include various combinations of ring, tip, coil, shocking electrodes, and the like.

The IMD 100 includes a programmable microcontroller 220 that controls various operations of the IMD 100, including cardiac monitoring and stimulation therapy. The microcontroller 220 includes a microprocessor (or equivalent control circuitry), one or more processors, RAM and/or ROM memory, logic and timing circuitry, state machine circuitry, and I/O circuitry. The IMD 100 further includes a ventricular pulse generator 222 that generates stimulation pulses for connecting the desired electrodes to the appropriate I/O circuits, thereby facilitating electrode programmability. The switch 226 is controlled by a control signal 228 from the microcontroller 220.

A pulse generator 222 is illustrated in FIG. 2A. Optionally, the IMD 100 may include multiple pulse generators, similar to the pulse generator 222, where each pulse generator is coupled to one or more electrodes and controlled by the microcontroller 220 to deliver select stimulus pulse(s) to the corresponding one or more electrodes. The IMD 100 includes sensing circuit 244 selectively coupled to one or more electrodes that perform sensing operations, through the switch 226 to detect the presence of cardiac activity in the chamber of the heart 111. The output of the sensing circuit 244 is connected to the microcontroller 220 which, in turn, triggers, or inhibits the pulse generator 222 in response to the absence or presence of cardiac activity. The sensing circuit 244 receives a control signal 246 from the microcontroller 220 for purposes of controlling the gain, threshold, polarization charge removal circuitry (not shown), and the timing of any blocking circuitry (not shown) coupled to the inputs of the sensing circuit 224.

In the example of FIG. 2A, the sensing circuit 244 is illustrated. Optionally, the IMD 100 may include multiple sensing circuits 244, where each sensing circuit is coupled to one or more electrodes and controlled by the microcontroller 220 to sense electrical activity detected at the corresponding one or more electrodes. The sensing circuit 224 may operate in a unipolar sensing configuration or a bipolar sensing configuration.

The IMD 100 further includes an analog-to-digital (A/D) data acquisition system (DAS) 250 coupled to one or more electrodes via the switch 226 to sample cardiac signals across any pair of desired electrodes. The A/D converter 250 is configured to acquire intracardiac electrogram signals, convert the raw analog data into digital data and store the digital data for later processing and/or telemetric transmission to an external device 600 (e.g., a programmer, local transceiver, or a diagnostic system analyzer). The A/D converter 250 is controlled by a control signal 256 from the microcontroller 220.

The switch 226 may be coupled to an LV lead having multiple LV electrodes, at least one of the LV electrodes configured to be located proximate to the LV site corresponding to the pacing site and to deliver the burst pacing therapy. The switch 226 may be further coupled to a second lead with at least one of a superior vena cava (SVC) coil electrode or an RV coil electrode, the shock vector including a CAN of the IMD and at least one of the SVC coil electrode or the RV coil electrode.

The microcontroller 220 is operably coupled to a memory 260 by a suitable data/address bus 262. The programmable operating parameters used by the microcontroller 220 are stored in the memory 260 and used to customize the operation of the IMD 100 to suit the needs of a particular patient. The operating parameters of the IMD 100 may be non-invasively programmed into the memory 260 through a telemetry circuit 264 in telemetric communication via communication link 266 (e.g., MICS, Bluetooth low energy, and/or the like) with the external device 600.

The IMD 100 can further include one or more physiological sensors 270. Such sensors are commonly referred to as “rate-responsive” sensors because they are typically used to adjust pacing stimulation rates according to the exercise state of the patient. However, the physiological sensor 270 may further be used to detect changes in cardiac output, changes in the physiological condition of the heart, or diurnal changes in activity (e.g., detecting sleep and wake states). Signals generated by the physiological sensors 270 are passed to the microcontroller 220 for analysis. While shown as being included within the IMD 100, the physiological sensor(s) 270 may be external to the IMD 100, yet still, be implanted within or carried by the patient. Examples of physiological sensors might include sensors that, for example, sense respiration rate, pH of blood, ventricular gradient, activity, position/posture, minute ventilation, and/or the like.

A battery 272 provides operating power to all of the components in the IMD 100. The battery 272 is capable of operating at low current drains for long periods of time, and is capable of providing a high-current pulses (for capacitor charging) when the patient requires a shock pulse (e.g., in excess of 2 A, at voltages above 2 V, for periods of 10 seconds or more). The battery 272 also desirably has a predictable discharge characteristic so that elective replacement time can be detected. As one example, the IMD 100 employs lithium/silver vanadium oxide batteries.

The IMD 100 further includes an impedance measuring circuit 274, which can be used for many things, including sensing respiration phase. The impedance measuring circuit 274 is coupled to the switch 226 so that any desired electrode and/or terminal may be used to measure impedance in connection with monitoring respiration phase. The IMD 100 is further equipped with a communication modem (modulator/demodulator) 240 to enable wireless communication with other devices, implanted devices and/or external devices. In one implementation, the communication modem 240 may use high frequency modulation of a signal transmitted between a pair of electrodes. As one example, the signals may be transmitted in a high frequency range of approximately 10-80 kHz, as such signals travel through the body tissue and fluids without stimulating the heart or being felt by the patient.

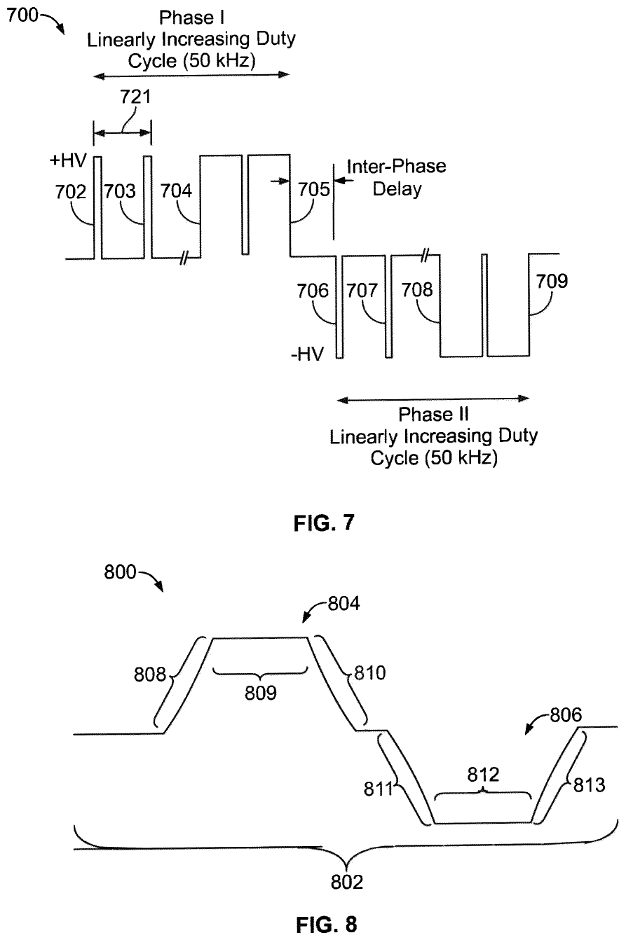

The microcontroller 220 further controls a shocking circuit 280 by way of a timing control 232. The shocking circuit 280 generates shocking pulses, such as MV shocks, LV shocks, etc., as controlled by the microcontroller 220. In accordance with some embodiments, the shocking circuit 280 includes a single change storage capacitor that delivers entire phase I and phase II therapies. The shocking circuit 280 is controlled by the microcontroller 220 by a control signal 282. Optionally, the microcontroller 220 may generate the control signals described in connection with FIGS. 7 and 8 to shape MV and LV shocks.

Although not shown, the microcontroller 220 may further include other dedicated circuitry and/or firmware/software components that assist in monitoring various conditions of the patient's heart and managing pacing therapies. The microcontroller 220 further includes a timing control 232, an arrhythmia detector 234, a morphology detector 236 and multi-phase VF therapy controller 233. The timing control 232 is used to control various timing parameters, such as stimulation pulses (e.g., pacing rate, atria-ventricular (AV) delay, atrial interconduction (A-A) delay, ventricular interconduction (V-V) delay, etc.) as well as to keep track of the timing of RR-intervals, refractory periods, blanking intervals, noise detection windows, evoked response windows, alert intervals, marker channel timing, and the like. The timing control 232 controls a timing for delivering the phase I, II and III therapies in a coordinated manner. The timing control 232 controls the phase II and III therapy timed relative to the MV shocks to cooperate with the MV shocks to terminate fibrillation waves of the ventricular arrhythmia episode and to reduce a defibrillation threshold of the heart below a shock-only defibrillation threshold.

The morphology detector 236 is configured to review and analyze one or more features of the morphology of CA signals. For example, in accordance with embodiments herein, the morphology detector 236 may analyze the morphology of detected R waves, where such morphology is then utilized to determine whether to include or exclude one or more beats from further analysis. For example, the morphology detector 236 may be utilized to identify non-conducted ventricular events, such as ventricular fibrillation and the like.

The arrhythmia detector 234 is configured to apply one or more arrhythmia detection algorithms for detecting arrhythmia conditions. By way of example, the arrhythmia detector 234 may apply various VF detection algorithms. The arrhythmia detector 234 is configured to declare a ventricular fibrillation (VF) episode based on the cardiac events.

The therapy controller 233 is configured to perform the operations described herein. The therapy controller 233 is configured to identify a multi-phase VF therapy based on the ventricular fibrillation episode, the multi-phase VF therapy including MV shocks, LV shocks and a pacing therapy. The therapy controller 233 is configured to manage delivery of the burst pacing therapy at a pacing site in a coordinated manner after the MV and LV shocks. The pacing site is located at one of a left ventricular (LV) site or a right ventricular (RV) site. The therapy controller 233 is configured to manage delivery of the MV shock along a shocking vector between shocking electrodes.

The therapy controller 233 is further configured to analyze a timing of VF beats to obtain at least one of a VF cycle length (CL) or variation and to determine at least one of a number of pulses in a pulse train of the burst pacing therapy or a duration of pulse train of the burst pacing therapy based on at least one of the VF cycle length or variation. The therapy controller 233 may be further configured to set a timing delay to time the burst pacing therapy such that one or more of pulses therefrom occur during a period of time in which a local tissue region surrounding the pacing site is excitable and not refractory. The therapy controller 233 may be configured to set a frequency of the burst pacing therapy at a high frequency relative to a cycle length of non-fibrillation arrhythmias.

In accordance with embodiments, the IMD 100 may represent a subcutaneous implantable cardioverter defibrillator (S-ICD). Optionally, the communication modem 240 may be configured to wirelessly communicate with a leadless pacemaker, such as to pass timing information there between. The S-ICD may deliver phase I and II therapies, while the phase III pacing therapy may be delivered by the S-CID or the leadless pacemaker. The communication modem 240 may transmit timing information to a leadless pacemaker such as when sending an instruction for the leadless pacemaker to deliver pacing therapies in connection with embodiments herein. The communication modem 240 may receive timing information from a leadless pacemaker such as when receiving a direction from the leadless pacemaker that the low voltage therapy has been delivered or is currently being delivered and that S-ICD should now deliver the HV shock(s).

### Subcutaneous Implantable Medical Device

FIG. 2B illustrates a graphical representation of an implantable medical system 261 that is configured to apply multi-phase VF therapy in accordance with embodiments herein. Embodiments may be implemented in connection with one or more subcutaneous implantable medical devices (S-IMDs). Non-limiting examples of S-IMDs include one or more of subcutaneous implantable cardioverter defibrillators (S-ICD). For example, the S-IMD may include one or more structural and/or functional aspects of the device(s) described in U.S. application Ser. No. 15/973,219, (docket A17E1043) titled “IMPLANTABLE MEDICAL SYSTEMS AND METHODS INCLUDING PULSE GENERATORS AND LEADS”, filed May 7, 2018; U.S. application Ser. No. 15/973,195,(docket A17E1045) titled “SUBCUTANEOUS IMPLANTATION MEDICAL DEVICE WITH MULTIPLE PARASTERNAL-ANTERIOR ELECTRODES”, filed May 7, 2018; which are hereby incorporated by reference in their entireties.

The system 261 includes a subcutaneous implantable medical device (S-IMD) 263 that is configured to be implanted in a subcutaneous area exterior to the heart. The S-IMD 263 is positioned in a subcutaneous area or region, and more particularly in a mid-axillary position along a portion of the rib cage 275. Optionally, the system 261 may also include a leadless pacemaker 269 implanted within the heart, such as at an apex 271 of the right ventricle. Optionally, the leadless pacemaker 269 may be omitted entirely. The system 261 does not require insertion of a transvenous lead.

The pulse generator 265 may be implanted subcutaneously and at least a portion of the lead 267 may be implanted subcutaneously. In particular embodiments, the S-IMD 263 is an entirely or fully subcutaneous S-IMD. Optionally, the S-IMD 263 may be positioned in a different subcutaneous region.

The S-IMD 263 includes a pulse generator 265 and at least one lead 20 that is operably coupled to the pulse generator 265. The lead 267 includes at least one electrode segment 273 that is used for providing MV shocks for defibrillation. Optionally, the lead 267 may include one or more sensing electrodes. The pulse generator 265 includes a housing that forms or constitutes an electrode utilized to deliver MV shocks. The electrode associated with the housing of the pulse generator 265 is referred to as the “CAN” electrode.

In an alternative embodiment, the lead 267 may include one or more electrode segments, in which the electrode segments are spaced apart from one another having an electrical gap therebetween. The lead body may extend between the gap. One electrode segment may be positioned along an anterior of the chest, while another electrode segment may be positioned along a lateral and/or posterior region of the patient. The electrode segments may be portions of the same lead, or the electrode segments may be portions of different leads. The electrode segments may be positioned subcutaneously at a level that aligns with the heart of the patient for providing a sufficient amount of energy for defibrillation. The lead includes a lead body that extends from the mid-auxiliary position along an inter-costal area between ribs and oriented with the coil electrode(s) extending along the sternum (e.g., over the sternum or parasternally within one to three centimeters from the sternum). A proximal end the coil electrodes may be located proximate to the xiphoid process.

### Coordination of Multi-phase HV Shock Therapy

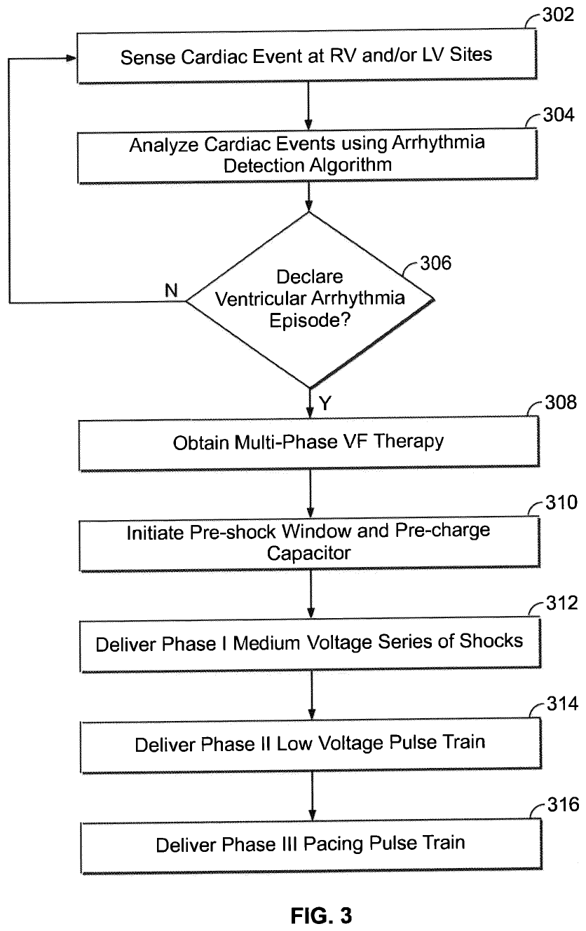

FIG. 3 illustrates a process for coordinating a multi-phase VF therapy that includes phase I, II, III therapies in accordance with embodiments herein. The process of FIG. 3 may be implemented by one or more processors within or distributed between an IMD, a S-IMD, leadless pacemaker, local external device and/or remote server.

At 302, cardiac events are sensed by electrodes located proximate to one or more sensing sites. For example, the electrodes may be located at one or more right ventricular (RV) sites, one or more left ventricular (LV) sites of the heart, one or more subcutaneous sites outside the heart and the like. The cardiac events are sensed over a period of time corresponding to a detection period. At 304, the one or more processors analyze the cardiac events utilizing various arrhythmia detection algorithms, such as algorithms that the detect bradycardia, fibrillation and/or tachycardia events.

At 306, the one or more processors determine whether to declare a ventricular fibrillation (VF) episode based on the analysis of the cardiac events. When a VF episode is not declared, flow returns to 302. Alternatively, when a VF episode is declared, flow continues to 308.

At 308, the one or more processors identify a multi-phase VF therapy to be delivered. The multi-phase VF therapy may be predefined, such as through programming by a clinician. Additionally or alternatively, the multi-phase VF therapy may be identified based on the nature and/or characteristics of the ventricular arrhythmia episode that was declared. As explained herein, the multi-phase VF therapy includes at least two and potentially three phases that are delivered in a coordinated manner, such as based on a predetermined timing relation there between. The predetermined timing relation between the phases of the multi-phase VF therapy is defined to change a condition of the heart to have a multi-phase defibrillation threshold, which is lower than the shock only defibrillation threshold of the heart that would otherwise be exhibited, but for the two or three phases of the therapy.

For example, the predetermined timing relation may be that the phase II and phase III therapies are delivered at predetermined times following delivery of the defibrillation MV shocks. Alternatively, the predetermined timing relation may be that the phase II therapy is started before completion of the defibrillation MV shocks but continues in a temporal overlapping manner with the defibrillation MV shock. Additionally or alternatively, the predetermined timing relation may be that the phase III therapy is delivered in a temporal overlapping manner during the phase II therapy and optionally during the phase I therapy.

The identification of the multi-phase VF therapy includes setting one or more therapy related parameters including one or more pacing related parameters, one or more low voltage shock related parameters, and one or more MV shock related parameters. Non-limiting examples of the pacing related parameters include the electrode combination to deliver the pacing pulses, a number of pacing pulses to be delivered during a pacing burst or pulse train, pulse width, pulse amplitude, pulse frequency (e.g., also referred to as the pulse to pulse or inter-pulse delay) and the like. Non-limiting examples of the LV and MV shocks related parameters include the electrode combination to deliver the shocks, a number of pulses to be delivered in the shock, shock pulse width, shock pulse amplitude, shock pulse frequency (e.g., also referred to as the shock pulse to pulse or shock inter-pulse delay) and the like. While the operation for identifying the multi-phase AF therapy is illustrated at 308 in the process of FIG. 3, it is understood that the multi-phase AF therapy may be preprogrammed and/or identified at various other points in time. For example, the multi-phase AF therapy may be identified before, during or after the sensing of cardiac events at 302. Optionally, the multi-phase AF therapy may be identified before, during or after the analysis of the cardiac events at 304, before, during or after the declaration at 306. Optionally, the multi-phase AF therapy may be identified and programmed at a point in time entirely separate from the operations of FIG. 3.

At 310, the one or more processors initiate a pre-shock window and begin pre-charging the single charge storage capacitor used to deliver the phase I therapy, and phase II therapies. The processors direct the charging circuit to charge the single charge storage capacitor (in some embodiments) and/or capacitor bank (in other embodiments) to a full voltage of up to 500 V, and more preferably between 400 V and 475 V. Additionally or alternatively, the charging circuit may charge the single charge storage capacitor to a full charge of no more than 25 J. Additionally, the one or more processors monitor a multi-phase coordination interval in connection with the predetermined coordinated timing between the phase I-III therapies.

At 312, the one or more processors deliver the phase I therapy. The phase I therapy includes a combination of two or more medium voltage shocks that, in accordance with at least some embodiments are delivered entirely from a single charge storage capacitor. Non-limiting examples of MV shock related parameters include the electrode combination that delivers the MV shock, the polarity designation between cathode and anode electrodes within the electrode combination, shock voltage, pulse width, a number of phases within the shock (e.g., monophasic, biphasic, tri-phasic) and the like. By way of example, the combination may include two or three medium voltage shocks delivered within a cycle length of a single VF episode. In accordance with some embodiments, an entirety of the therapy delivered during the first phase is delivered from the single charge storage capacitor based on a single charge of the capacitor. The MV shocks represent a defibrillation stimulation delivered at a select energy level that is at least one of i) no more than 25 J or ii) has a maximum voltage of no more than 500V. As a further example, the select energy level may be at least one of i) between 15 J-25 J or ii) has a maximum voltage of between 100V-475V for an IMD with a transvenous lead. Once the single charge storage capacitor is fully charged, the single charge storage capacitor delivers, during the phase I therapy, the combination of two or more MV shocks at first and second voltage levels, respectively, below the fully charged voltage level. The processors may manage termination of the MV shocks based on one or more of shock pulse with, a trailing edge voltage of the shock waveform and/or an energy content of each biphasic shock. The processors manage termination of the MV shocks in an appropriate manner such that the single charge storage capacitor retains a desired post phase I voltage following an end of the first phase. Additionally or alternatively, the charging circuit may charge the capacitor to a full charge of no more than 25 J and manage energy discharge from the capacitor, during the phase I therapy, to retain a select post phase I residual charge.

In accordance with at least some embodiments, the phase I therapy is delivered along a far field shock vector between an RV electrode and the IMD housing/CAN electrode. Optionally, the shock vector may comprise a combination of shock vectors, such as between the IMD housing and a virtual or collective electrode defined by i) one or more electrodes in the RV and ii) one or more electrodes in the AV. Optionally, the shock vector may comprise a combination of shock vectors, such as between the IMD housing and a virtual or collective electrode defined by i) one or more electrodes in the RV and ii) one or more electrodes proximately to the LV. Optionally, when implemented in connection with a subcutaneous IMD, the phase I therapy may be delivered i) between one or more parasternal electrodes and a subcutaneous IMD housing/CAN electrode, ii) between one or more subcutaneous electrodes, iii) between one or more subcutaneous electrodes and the IMD housing/can electrode, and the like.

At 314, the one or more processors deliver the phase II therapy. The phase II therapy includes a pulse train of low voltage pulses that are delivered at least partially from the same single charge storage capacitor as used to deliver the phase I therapy. In accordance with at least some embodiments, the train of low voltage pulses are delivered entirely from the same single charge storage capacitor as used to deliver the phase I therapy. By way of example, the phase II therapy may include six or more low voltage pulses delivered at a voltage that is slightly higher than the ventricular capture threshold. The train of low voltage pulses (e.g., six pulses) are delivered within an interval that is shorter than a single cycle length of the fibrillation episode. During the phase II therapy, the single charge storage capacitor delivers the low voltage pulse train at a desired low voltage which is below the post phase I voltage retained by the capacitor at the end of the phase I therapy.

Optionally, the single charge storage capacitor may deliver all of the phase I therapy and a portion of the phase II therapy based on a single charge, while either i) a second capacitor delivers a remainder of the phase II therapy or ii) the single charge storage capacitor is recharged during the phase II therapy and then used to deliver the remainder of the phase II therapy.

In accordance with at least some embodiments, the phase II therapy is delivered along the same far field shock vector(s) as the phase I therapy. Additionally or alternatively, the phase II therapy may be delivered along one or more other shock vectors, separately designated as a low voltage shock vector, such as between an RV electrode and the IMD housing/can electrode. Optionally, the low voltage shock vector may comprise a combination of shock vectors, that differs from the phase I shock vectors such as between the IMD housing and a different virtual or collective electrode defined by i) one or more electrodes in the RV and ii) different one or more electrodes in the AV. Optionally, the low-voltage shock vector may comprise a combination of shock vectors that differs from the phase I shock vectors, such as between the IMD housing and a different virtual or collective electrode defined by i) one or more electrodes in the RV and ii) one or more electrodes proximately the LV. Optionally, when implemented in connection with a subcutaneous IMD, the phase II therapy may be delivered i) between one or more parasternal electrodes and a subcutaneous IMD housing/can electrode, ii) between one or more subcutaneous electrodes, iii) between one or more subcutaneous electrodes and the IMD housing/can electrode, and the like. Optionally, the phase II therapy may be delivered along a different shock vector, then the shock vector utilized in the phase I therapy.

At 316, the one or more processors deliver the phase III therapy. The phase III therapy includes a series of pacing pulses that are delivered from a pacing storage device. Optionally, the series of pacing pulses may be delivered entirely from the same single charge storage capacitor as used to deliver the phase I and phase II therapies, wherein the single charge storage capacitor delivers the series of pacing pulses at a pacing voltage which is below both the post phase I voltage and post phase II voltage retained by the capacitor at the end of the phase I therapy and phase II therapy, respectively. Optionally, the phase III therapy may be delivered by a separate pacing pulse generator separate and independent from the single charge storage capacitor utilized to deliver the MV shock and low-voltage shock.

In accordance with at least some embodiments, the phase III therapy is delivered between a same far field vector (e.g., between an RV electrode and the IMD housing/can electrode), as utilized to deliver the phase I therapy and/or the phase II therapy. Optionally, when implemented in connection with the subcutaneous IMD, the phase III therapy may be delivered between one or more parasternal electrodes and a subcutaneous IMD housing/can electrode, between one or more subcutaneous electrodes, between one or more subcutaneous electrodes and an IMD housing/can electrode, and the like. For example, the phase III therapy may be delivered along a vector between an SVC electrode and an RV electrode, between RV and one or more LV electrodes, between an SVC electrode and one or more LV electrodes, and the like. Optionally, the phase III therapy may be delivered by a leadless pacemaker separate from the phase I therapy and/or phase II therapy.

By way of example, the third phase may include a series of pacing pulses delivered at a high rate and/or appropriately timed from a sensed intrinsic event. The sequence of multiple shocks at medium voltage and low voltage, along with the high-frequency pacing pulses lowers a total energy required to terminate a ventricular fibrillation, as compared to a traditional high-voltage fibrillation shock delivered alone.

Optionally, the method of FIG. 3 may include additional sensing and arrhythmia detection operations between 312 and 314 that are utilized as a secondary determination as to whether the next phase of the therapy should be delivered. For example, cardiac events are sensed by electrodes located proximate to one or more sensing sites. For example, after delivery of the phase I therapy at 312 and before delivery of the phase II therapy at 314, one or more additional cardiac events may be sensed by electrodes located at one or more right ventricular (RV) sites, and/or one or more left ventricular (LV) sites of the heart, and/or one or more subcutaneous sites outside the heart and the like. The additional cardiac events are analyzed using various arrhythmia detection algorithms to determine whether the ventricular arrhythmia episode (detected at 306) continues or has been terminated by the phase I therapy. When the ventricular arrhythmia episode is sensed after 312 and determined to be ongoing, in response thereto, the phase II therapy is delivered at 314. Alternatively, when the ventricular arrhythmia episode is identified to have been terminated by the phase I therapy, the one or more processors cancel or terminate subsequent delivery of the phase II therapy and phase III therapy.

Additionally or alternatively, an additional sensing and arrhythmia detection operation may be performed between 314 and 316 as a secondary determination as to whether the phase III therapy should be delivered. For example, cardiac events are sensed by electrodes located proximate to one or more sensing sites. For example, after delivery of the phase II therapy at 314 and before delivery of the phase III therapy at 316, one or more additional cardiac events may be sensed by electrodes located at one or more right ventricular (RV) sites, and/or one or more left ventricular (LV) sites of the heart, and/or one or more subcutaneous sites outside the heart and the like. The additional cardiac events are analyzed using various arrhythmia detection algorithms to determine whether the ventricular arrhythmia episode (detected at 306) continues or has been terminated by the phase I and phase II therapies. When the ventricular arrhythmia episode is sensed after 314 and determined to be ongoing, in response thereto, the phase III therapy is delivered at 316. Alternatively, when the ventricular arrhythmia episode is identified to have been terminated by the phase II therapy, the one or more processors cancel or terminate subsequent delivery of the phase III therapy.

Optionally, based on the additional cardiac events sensed and analyzed, the multiphase therapy may jump from the phase I therapy at 312 to the phase III therapy at 316, and omit entirely the phase II therapy at 314.

Optionally, the sensing and arrhythmia detection operations performed between 312 and 314, and between 314 and 316, may also include updating one or more parameters of the therapy to be delivered during the corresponding phase. For example, the additional cardiac events sensed after 312 may be analyzed and utilized to identify a change in one or more parameters of the phase II therapy. For example, amplitude, pulse width or pulse interval may be adjusted between the low-voltage pulse train delivered in the phase II therapy. Additionally or alternatively, the additional cardiac events sensed after 314 may be analyzed and utilized to identify a change in one or more parameters of the phase III therapy. For example, amplitude, pulse width or pulse interval may be adjusted between the pacing pulse train of the phase III therapy.

Optionally, the parameters of the phase I, II, and/or III therapies may be adjusted in accordance with the methods and systems described in U.S. Pat. No. 8,060,200, to Hofstadter et al., titled “SELF-ADJUSTING OPTIMAL WAVEFORMS”, issuing Nov. 15, 2011 (attached at appendix A), the complete subject matter of which is incorporated herein by reference in its entirety. Optionally, the parameters of the phase I, II, and/or III therapies may be adjusted in accordance with the DeFT Response™ process implemented today on various ICDs, as described in brochure attached at appendix A, the complete subject matter of which is incorporate herein by reference in its entirety. By way of example, the MV shocks delivered in the phase I therapy may be managed and delivered based on the methods and systems described in the '200 patent and Deft Brochure at Appendix A, although it is recognized that the membrane time constant may change based on the peak voltage level and capacitance utilized to form the MV shocks.

Conceptually, the heart may be viewed as a collection of reentrant circuits that carry ventricular fibrillation waves. In accordance with embodiments herein, the multi-phase VF therapy affords a greater chance of capturing ventricular fibrillation waves as such fibrillation waves propagate through different regions of the heart, thereby terminating an associated number of reentrant circuits. In accordance with embodiments herein, the frequency of the pacing pulses may be set substantially higher than a frequency associated with VT or other non-fibrillation arrhythmias. Thus, one or more of the pulses in the burst pacing therapy will be delivered between successive points in time when the local tissue region transitions to the excitable state due to the VF episode. For example, the pacing related parameters may define the burst pacing to have a high frequency, such as at least 30 Hz, or preferably a frequency between 50 and 100 Hz, or more preferably at 50 Hz. Additionally or alternatively, the pacing related parameters may be defined in terms of the pulse to pulse (interpulse) separation between successive pacing pulses, such as no more than 30 ms, and as another example between 15 and 25 ms, and more preferably 20 ms. The pacing related parameters may define the pulse duration, such as to be 0.3 to 1.0 ms, or 0.4 to 0.8 ms, or more preferably 0.5 ms. by way of example, the burst pacing therapy may include one or more burst, where each burst delivers the pacing pulses for 0.5 seconds to 2 seconds. The pacing related parameters may define the pacing pulses to be unipolar or bipolar. In at least certain embodiments, it may be preferable to use unipolar pacing pulses. As one particular configuration, the burst parameters may be a 50 Hz high frequency pacing pulses (20 ms inter-pulse separation), and with unipolar stimulation having pulse durations of 0.5 ms. The pacing related parameters may designate a combination of LV electrodes to simultaneously deliver the pacing pulses. As a further example, when an LV lead includes four electrodes, the pacing related parameters may designate a combination of one or more of the LV electrodes to deliver the burst pacing therapy (e.g., P1, M2, M3 and D1 electrodes simultaneously deliver each pacing pulse). Alternatively, a subset of the LV electrodes may be utilized to deliver the burst pacing therapy. Additionally or alternatively, the burst pacing therapy may include delivery of separate pacing pulse bursts at separate combinations of LV electrodes.

The locations, at which the phase I, phase II and phase III therapies are delivered, is dependent upon the overall electrode configuration. For example, in accordance with embodiments herein, when an MPP lead is located along the LV, the phase II and/or phase III therapies may be delivered at one or more LV electrodes.

Optionally, following delivery of the phase 3 therapy at 316, the one or more processors may return to 302 and since new cardiac events, analyze the cardiac events and determine whether the VT episode was terminated. When the VT episode is not terminated, during a second iteration through the operations of FIG. 3, when treating a same VT episode, the one or more processors may utilize the same multiphase VF therapy. Additionally or alternatively, the one or more processors may modify the multiphase VF therapy during a second attempt to terminate the same VT episode. Additionally or alternatively, following 316, when the one or more processors determined that the VT episode has not terminated, the one or more processors may revert to a higher voltage VT therapy, in which one or more high-voltage shocks are delivered. As one example, after 316, when a VF episode continues, the processors may direct one or more capacitors to be pre-charged to a high voltage (e.g., 800 V-900 V) to just deliver a high energy shock.

Additionally or alternatively, the IMD may include a bank of two or more capacitors that are connected in parallel during the operations of FIG. 3. When the two or more capacitors are connected in parallel, the bank of capacitors would be configured to deliver a medium voltage series of shocks, followed by the low voltage pulse train.

Additionally or alternatively, when utilizing a bank of two or more capacitors connected in parallel, following the delivery of the phase 3 therapy at 316, in the event the VF episode is not terminated, the one or more processors may direct a capacitor coupling circuit to reconnect the capacitors in series in order to deliver one or more high-voltage shocks. For example, when the bank of capacitors are connected in parallel, the capacitors may have a maximum voltage of up to 500 V (thereby being configured to deliver medium voltage shocks), but when reconnected in series, the capacitors may afford a maximum voltage of 800 V-900 V (thereby being configured to deliver high-voltage shocks).

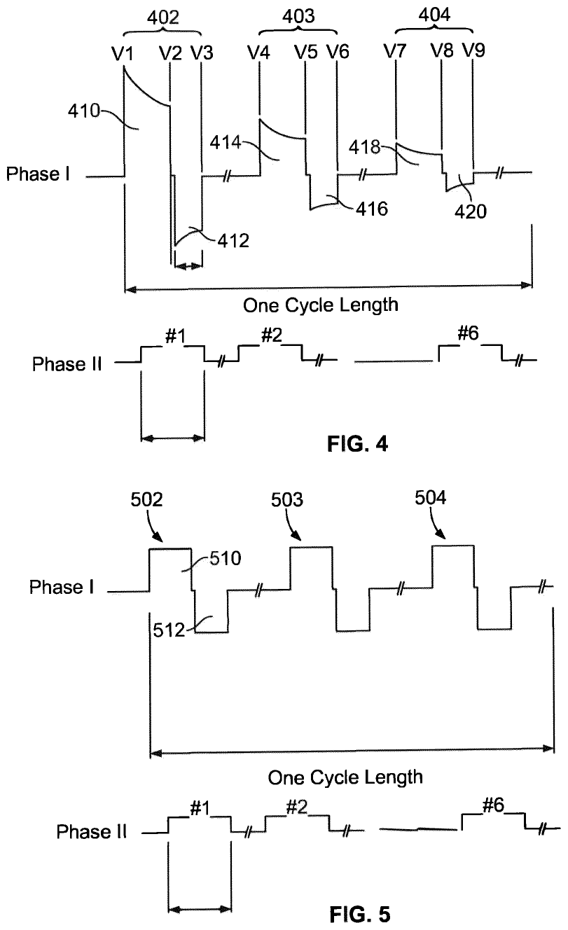

FIG. 4 illustrates example waveforms that may be delivered during the phase I and phase II therapies in accordance with embodiments herein. The phase III therapy includes a series of pacing pulses that need not be illustrated by separate Figure. During the phase I therapy, three biphasic MV shocks 402-404 are delivered, separated from one another by a predetermined pulse to pulse interval. The MV shocks 402-404 exhibit waveforms that have a decaying voltage over the course of each phase segment. The first MV shock 402 includes positive and negative phase segments 410, 412, each of which exhibits a certain amount of decay between the leading and trailing edge voltages. The positive phase segment 410 includes a leading-edge voltage V1 and a trailing edge voltage V2 that is lower than V1, while the negative phase segment 412 includes a leading-edge voltage V2 and a trailing edge voltage V3. The difference in voltage between the leading and trailing edges of each phase segment will vary dependent upon certain factors such as the capacitance of the MV shocking capacitor, a load resistance experienced by the shocking circuit across the patient's heart, the electrode configuration, the pulse width of the phase segment and the like. The leading-edge voltage of the negative phase segment 412 is substantially similar to the trailing edge voltage of the positive phase segment 410. After a desired delay, the second MV shock 403 is delivered with positive and negative phase segments 414, 416. The positive phase segment 414 has a leading-edge voltage V4 and a trailing edge voltage V5 that is lower than V4. The leading-edge voltage V4 of the positive phase segment 414 of the second MV shock 403 has substantially the same voltage as the trailing-edge voltage V3 of the negative phase segment 412 of the first MV shock 402. The leading-edge voltage of the negative phase segment 416 has substantially the same voltage, V5, as the trailing edge voltage of the positive phase segment 412. The negative phase segment 416 terminates at a trailing edge having a voltage V6. Similarly, the third MV shock 404 has positive and negative phase segments 418, 420 with leading and trailing edge voltages V7-V9. The leading-edge voltage V7 of the positive phase segment 418 of the third MV shock 404 has substantially the same voltage as the trailing-edge voltage V6 of the negative phase segment 416 of the second MV shock 403.

The MV shocks 402-404 are delivered from the same single charge storage capacitor and are spaced apart from one another by a relatively close amount of time, such that, in some embodiments, it is not possible to recharge the single charge storage capacitor between the MV shocks 402 and 403, or between the MV shocks 403 and 404. In conventional systems, each of the HV shocks would be delivered by a separate capacitor, which would also require a separate charging circuits and switching circuits, thereby causing the size of the IMD to become undesirably large. In accordance with embodiments herein, an overall size of the IMD is reduced, and an amount of circuitry and other components are simplified, by utilizing a single charge storage capacitor to deliver all of the MV shocks 402-404 in the phase I therapy based on a single charge of the single charge storage capacitor. In accordance with new and unique aspects herein, as illustrated in Table 1 below, the voltages V3 and V4 between successive MV shocks are substantially similar due to the use of a single charge storage capacitor to deliver both of the first and second MV shocks 402 and 403 during a single charge operation (e.g., without recharging the single charge storage capacitor between the first and second MV shocks). Similarly, in accordance with new and unique aspects herein, when a third MV shock 404 is utilized, the voltages V6 and V7 between successive MV shocks are substantially similar due to the use of a single charge storage capacitor to deliver both of the second and third MV shocks 403 and 404 during a single charge operation (e.g., without recharging the single charge storage capacitor between the first and second MV shocks).

Table 1 below sets forth example capacitances, pulse widths (PW 1, PW2) and voltages that may be utilized to form the shocking pulses 402-404 of the phase I therapy. It is recognized that the values in Table 1 are nonlimiting examples and that numerous alternatives may be used. As a nonlimiting example, when a single charge storage capacitor is utilized having a capacitance of 220 μF, and when the phase I therapy is defined to have first and second pulse widths PW1, PW2 of 5 ms and 2.5 ms for the positive and negative phase segments of each of the shocks 402-404, the voltages V1-V9 will be substantially as noted below in Table 1. As another nonlimiting example, when a single charge storage capacitor is utilized having a capacitance of 300 μF, and when the phase I therapy is defined to have first and second pulse widths PW1, PW2 of 5 ms and 2.5 ms for the positive and negative phase segments of each of the shocks 402-404, the voltages V1-V9 will be substantially as noted below in Table 1.

TABLE 1

| Capacitance/PW1/PW2 | V1 | | V2 | | V3 | | V4 | | V5 | | V6 | | V7 | | V8 | | V9 | |
|---|---|---|---|---|---|---|---|---|---|---|---|---|---|---|---|---|---|---|
| 220 uF/5 ms/2.5 ms | 450 | V | 285 | V | 228 | V | 220 | V | 144 | V | 115 | V | 110 | V | 73 | V | 58 | V |
| 300 uF/5 ms/2.5 ms | 450 | V | 322 | V | 273 | V | 265 | V | 195 | V | 165 | V | 160 | V | 118 | V | 100 | V |
| 220 uF/5 ms/2.5 ms | 450 | V | 261 | V | 203 | V | 195 | V | 113 | V | 85 | V | 80 | V | 46 | V | 36 | V |
| 300 uF/5 ms/2.5 ms | 450 | V | 287 | V | 247 | V | 240 | V | 153 | V | 132 | V | 127 | V | 81 | V | 70 | V |

FIG. 4 also illustrates a phase II therapy that includes a series of six low voltage pulses #1-#6, each of which has a select pulse width (e.g., 10 ms) and select amplitude (e.g., between 10 V and 20 V). While it is recognized that alternative pulse widths and amplitudes may be utilized for the low-voltage pulses (as well as a different number of low-voltage pulses), the pulse width and pulse amplitude, for the phase II therapy, are substantially greater than a conventional pacing pulse width and amplitude.

FIG. 5 illustrates example waveforms that may be delivered during the phase I and phase II therapies in accordance with an alternative embodiment. During the phase I therapy of FIG. 5, three biphasic MV shocks 502-504 are delivered, separated from one another by a predetermined pulse to pulse interval. Each of the MV shocks 502-504 have positive and negative phase segments. The waveforms of FIG. 5 differ from the waveforms in the embodiment of FIG. 4 in that the shape of the positive and negative phase segments in the waveforms in FIG. 5 correspond to a square non-decaying waveform that does not exhibit any decay in voltage over the course of the positive phase segment or the negative phase segment. By way of example, the MV shock 502 maintains a constant voltage (e.g., 100 V) over a positive phase segment 510. The MV shock 502 maintains the same constant voltage or a lower constant voltage over the negative phase segment 512.

Table 2 below sets forth example capacitances, pulse widths (PW1, PW2) and voltages that may be utilized to form the shocking pulses 502-504 of the phase I therapy. It is recognized that the values in Table 2 are nonlimiting examples and that numerous alternatives may be used. As a nonlimiting example, when a single charge storage capacitor is utilized having a capacitance of 220 μF, and when the phase I therapy is defined to have first and second pulse widths PW1, PW2 of 5 ms and 2.5 ms for the positive and negative phase segments of each of the shocks 502-504, the voltage may be constant for all phase segments at 100 V. As another nonlimiting example, when a single charge storage capacitor is utilized having a capacitance of 300 μF, and when the phase I therapy is defined to have first and second pulse widths PW1, PW2 of 5 ms and 2.5 ms for the positive and negative phase segments of each of the shocks 502-504, the voltage may be constant for all phase segments at 125 V.

TABLE 2

| Capacitance/PW1/PW2 | Voltage |
|---|---|
| 220 uF/5 ms/2.5 ms | 100 V |
| 300 uF/5 ms/2.5 ms | 125 V |

The foregoing discussion of FIGS. 3-5 concern embodiments in which a single storage capacitor is utilized to deliver an entirety of the phase I therapy, and at least a portion of the phase II therapy. Next, the discussion turns to an output control circuit that may be utilized in connection with delivering effective stimulation waveforms within the multiphase therapy described in connection with FIGS. 3-5. However it should be recognized that the following discussion, concerning the effective stimulation waveforms and the output control circuitry, may be implemented in combination with embodiments utilizing more than a single storage capacitor. Instead, the effective stimulation waveforms and the output control circuitry described hereafter may be implemented in connection with a bank of two or more storage capacitors, and/or other conventional charge storage configurations.

### Output Control Circuit

Next, the discussion turns to example circuits that may be utilized to generate waveforms utilized during the phase I therapy alone, and/or in combination with waveforms utilized to generate the phase II and/or phase III therapies. Optionally, an output circuit system may be implemented as described in U.S. Pat. No. 7,450,995, titled “IMPLANTABLE CARDIAC STIMULATION DEVICE INCLUDING AN OUTPUT CIRCUIT THAT PROVIDES ARBITRARILY SHAPED DEFIBRILLATION WAVEFORMS”, filed Oct. 15, 2003 and issuing Nov. 11, 2008, the complete and entire subject matter of which is expressly incorporated herein by reference in its entirety.

The output control system includes an output configured to be connected to a lead, a charging circuit, a capacitor switchably coupled to the charging circuit, a switching circuit coupled between the capacitor and the output, and an output control circuit configured to generate a control signal that includes control pulses that control the switching circuit to switchably connect the capacitor to the output. The output control circuit is configured to vary a duty cycle of the control pulses in a manner that defines a shape of an effective waveform for one or more shocks. In accordance with embodiments herein, the shape of the effective waveform includes a positive phase segment that is defined by a collection of the control pulses, in which the duty cycle varies such that an initial portion of the collection includes a first duty cycle and a final portion of the collection includes a second duty cycle that is longer than the first duty cycle. In accordance with embodiments herein, the output control circuit is configured to vary the first and second duty cycles to define a leading edge profile having an ascending non-linear shape that transitions from a low voltage to a high voltage, and a steady-state profile at the high voltage for a duration of the positive phase segment. In accordance with embodiments herein, the output control circuit is configured to increase the duty cycle of the successive control pulses from an initial shorter duty cycle to a final longer duty cycle. In accordance with embodiments herein, the capacitor is configured to store a full charge with a voltage of at least 400V, and the output control circuit is configured to vary the duty cycle of the control pulses to define the effective waveform of a first shock to have a voltage of less than 300V. In accordance with embodiments herein, the capacitor represents a single charge storage capacitor that is configured to store a full charge with a voltage of no more than 500V, and the output control circuit is configured to vary the duty cycle of the control pulses to define the effective waveform for first and second shocks to be delivered from the single charge storage capacitor based on a single charge of the single charge storage capacitor. In accordance with embodiments herein, the capacitor represents a single charge storage capacitor that is configured to store a full charge with a voltage of no more than 500V, and the output control circuit is configured to vary the duty cycle of the control pulses to deliver a multi-phase VF therapy that includes first and second phase therapies, wherein: a) during the first phase therapy, a combination of two or more medium voltage (MV) shocks are delivered entirely from the single charge storage capacitor; and b) during the second phase therapy, a low voltage pulse train is delivered at least partially from the single charge storage capacitor.

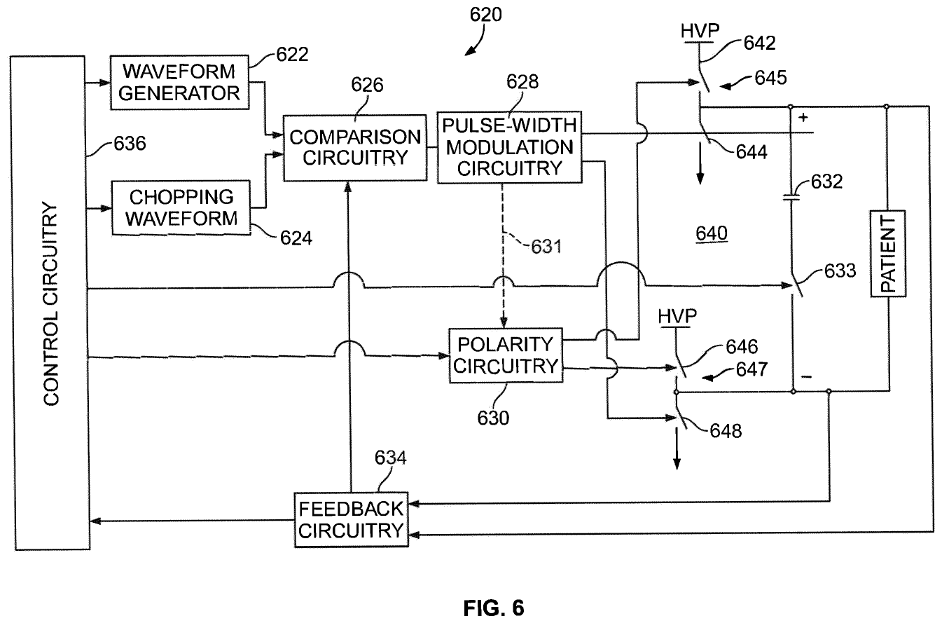

FIG. 6 illustrates a block diagram of an output control circuit formed in accordance with embodiments herein. By way of example, the output control circuit 620 may be provided as part of, or separate from, the shocking circuit 280 (FIG. 2A), and/or as part of, or separate from, the microcontroller 220 and VF therapy control 233. The output control circuit 620 is configured to provide a stimulation output having a waveform of substantially any desired shape. The output control circuit 620 may be utilized to manage delivery of MV shocks and low-voltage shocks in accordance with the multiphase therapy described herein. The output control circuit 620 avoids problems experienced by conventional output control circuits. For example, a conventional output control circuit would utilize high-voltage amplifiers (designed to operate with maximum charge storages of 800 V-900 V), and such high-voltage amplifiers would dissipate a substantial amount of energy/heat into the IMD during shock delivery. Thus, conventional high-voltage amplifiers were very energy inefficient and would lead to heating of the IMD. In accordance with embodiments herein, the output control circuit described as connection with FIG. 6, is more energy efficient and avoids excessive heating, as compared to conventional high-voltage circuits.

Additionally or alternatively, the output control circuit 620 may be utilized to manage delivery of only one of a low-voltage shock therapy, MV shock therapy and/or high voltage shock therapy, delivered as a single phase therapy. For example, the output control circuit 620 may be utilized to manage delivery only for a shock resembling the shape in phase I of FIG. 4 or FIG. 5. Separate and apart therefrom, the output control circuit 620 may be utilized to manage delivery only for a shock resembling the shape of phase II of FIG. 4 or FIG. 5. The control circuit 620 generally includes a waveform generator 622, a chopping waveform generator 624, a comparison circuit 626, a pulse-width modulation circuit 628, and a polarity control circuit 630. The control circuit 620 further includes an H-bridge 640, an output capacitor 632 and series switch 633, and a feedback circuit 634. The waveform generator 622 and chopping waveform generator 624 are coupled to the comparison circuit 626. The output of the comparison circuit 626 is coupled to the pulse modulation circuit 628. A connection 631 may be made between the pulse-width modulation circuit 628 and the polarity control circuit 630 to assure that polarity is not changed when the pulse-width modulation circuit is active.

The outputs of the pulse-width modulation circuit 628 and the polarity control circuit 630 are coupled to the H-bridge 640. The H-bridge 640 includes a first leg or half 645 and a second half 647. The first half 645 includes a pair of switching devices 642 and 644. The second half 647 of the H-bridge 640 includes switching devices 646 and 648. The switching devices may be, for example, IGBT devices. Switching devices 642 and 646 control the polarity of the stimulation output and devices 644 and 648 control the waveform of the stimulation output. The output capacitor 632 is coupled across the H-bridge 640. It in turn is coupled in parallel to the patient. The capacitor 632 is preferably a bipolar capacitor. The feedback circuit 634 is coupled across the capacitor 632 for sensing the output pulse voltage. The output of the feedback circuit 634 is coupled to the comparison circuit 626.

Switch 633 is in series with the capacitor 632. The capacitor 632 is coupled across the circuit output. The switch 633 may be employed for generation of a standard truncated exponential output. It also prevents charging of capacitor 632 during external defibrillation. In operation, the waveform generator 622 provides an electrical signal representing the desired waveform for the stimulation output, such as a defibrillation pulse. The chopping waveform generator 624 provides a high frequency (for example 500 kilohertz) triangle or saw-tooth waveform. The output of the comparison circuit 626 provides a control signal in response to the signals from the waveform generator 622 and the chopping waveform generator 624. The pulse-width modulation circuit 628 then provides a modulated output comprising a pulse train of pulses having varying duty cycles. The pulse-width modulation circuit further conditions the pulse train to adjust duty cycle, polarity, etc. The pulse-width modulated pulse train is coupled to switching devices 644 and 648 to control the waveform of the stimulation output. The control circuitry 636 provides a polarity control signal which is in turn provided to the polarity control circuit 630. The outputs of the polarity control circuit 630 are coupled to switching devices 642 and 646 for controlling the polarity of the defibrillation output pulse.

If the polarity is as indicated in FIG. 6, the voltage to the patient will be considered positive when switching device 642 is turned on, switching devices 644 and 646 are turned off, and when switching device 648 modulates the output voltage (HVP) to control the amplitude of the output voltage. Conversely, the voltage to the patient will be considered negative when switching device 646 is turned on, switching devices 642 and 648 are turned off, and switching device 644 modulates the output voltage to control the amplitude of the output voltage. The output amplitude is fed back to the comparison circuit 626 to adjust the pulse widths of the output based upon the desired amplification at the output. The feedback circuit 634 may, for example, be a differential amplifier. By way of example, during delivery of a single phase therapy and/or multiphase therapy, the feedback circuit 634 may continuously since the voltage level on the storage capacitor(s) 632 and provide the sensed voltage to the control circuitry 636 and comparison circuitry 626. The control circuitry 636 and/or comparison circuitry 626 adjust a duty cycle of control pulses within the control signals delivered to the switching devices 642-648 to maintain an effective waveform shape within the therapy. In the foregoing manner, the feedback circuitry 634 senses voltage drops across the storage capacitor(s) 632 and the control circuitry 636 and/or comparison circuitry 626 compensates for the drop in voltage by extending the duty cycle of individual control pulses (e.g., increasing the duty cycle from 10%, to 20%, 30%and upward toward 100%).

The pulse-width modulation circuit provides further conditioning of the modulation signal. For example, it may limit duty cycle or pulse train polarity based on output polarity. One advantage of the output circuit of FIG. 6 is that the amplitude control devices are switched at a sufficiently high frequency such that the patient, in conjunction with charge storage capacitor 632, will act as a low pass filter. More generally, the cells of the myocardium perform a low-pass filtering function in that the cells of the myocardium only experience an average of the energy/voltage delivered, and thus provide a virtual smoothing of the instantaneous voltage delivered at the electrodes over the course of an individual MV shock or low-voltage shock. As a result, high efficiency pulse-width modulation amplification is obtained without the use of a low pass inductor. This is particularly important in as much as space and in an implanted device is at a premium and inductors are generally large in size. It may also be noted that only switching devices 644 and 648 are switched to control the waveform of the output pulse. This may serve to lower the amount of energy required to operate the H-bridge and thus save battery power.

By way of example, the control circuit of FIG. 6 may implement a “high voltage class D amplifier design” that switches state based on a series of pulse-width modulated high frequency control pulses. The control circuit connect the energy source (e.g., capacitor 632) to the shocking electrodes for a series of pulse width modulated high-frequency switching states that corresponds to when the control pulses are in the ON or high state. The pulse width modulated high-frequency switching operations deliver energy through the shocking electrodes in a manner that allows the heart to integrate the energy over time to form an effective stimulation waveform that is realized to the heart.

The control pulses are pulse width modulated based on a duty cycle of each corresponding control pulse. For example, a control pulse series may be defined to have a select frequency, such as 50,000 cycles per second. During one cycle, the control pulse cycles transitions from an OFF or low state to an ON or high state and back to the OFF or low state. The duty cycle represents the percentage of time, over one cycle, that the control pulse remains in the ON or high state. The duty cycle may be expressed as a percentage of the period. For example, when the control signal has a frequency of 50 kHz, the period for one control pulse would be 0.02 msec. In accordance with at least some embodiments, a frequency of 50 kHz or higher for the control signal is desirable, as myocardial cells exhibit the low-pass filtering or smoothing characteristics described herein when exposed to instantaneous voltages having a period of less than or equal to 0.02 ms (corresponding to a frequency of 50 kHz). It is recognized that in some instances, a frequency below 50 kHz, such as 30 kHz-50 kHz, may be utilized depending upon the filtering/smoothing characteristics of the local myocardial cells. Continuing the foregoing example, when the duty cycle is 20%, one control pulse would remain in an ON or high state for 20% of 0.02 msec or 0.004 msec. In accordance with embodiments herein, the duty cycle of control pulses are pulse width modulated to control the shape of the effective stimulation waveform realized by the heart.

FIG. 7 illustrates an example of a control signal to manage the switches within the control circuit of FIG. 6 in accordance with an embodiment herein. The control signal 700 includes a series of control pulse 702-709 that are pulse width modulated. The control signal 700 has a frequency that defines a period extending from leading edges of successive control pulses 702-709 (denoted as period 721 between control pulses 702, 703). The period remains the same for the remaining control pulses 703-709. At least the control pulses 702, 703 occur earlier within a positive phase segment of a shock (at the lead edge profile), while at least the control pulses 704, 705 occur later within the positive phase segment of the shock (at the trailing edge profile). It is recognized that a large number of control pulses are not shown, but occur between the control pulses 703, 704 at a time that corresponds to the intermediate portion of the positive phase segment. The pulse widths of the control pulses may be widened to add energy to the delivered shock.

The control signal 700 is delivered to one or more of the switching devices 642, 644, 646 and 648 to modulate the output voltage. The control pulses 702-709 have different pulse widths (duty cycles) that are varied to achieve a desired waveform shape for an effective stimulation waveform. The control signal 700 directs the output control circuit (FIG. 6) to switch with sufficiently high frequency such that the patient experiences an MV shock having a desired amplitude and shape. As one example, the control signal 700 may utilize varying duty cycles between control pulses 702-709 to achieve effective stimulation waveforms such as, but not limited to, the shapes illustrated in the phase I and/or phase II therapies of FIG. 4, the shapes illustrated in the phase I and phase II therapies of FIG. 5, the shape illustrated in the therapy of FIG. 8, and the like. Additionally or alternatively, the control signal 700 may be configured to manage the switching devices 642-648 to produce an effective stimulation waveform having a constant voltage amplitude (e.g., 100 V-300 V), while the instantaneous voltage charge on the shocking capacitor(s) 632 drops or reduces over the course of an individual shock. As a nonlimiting example, the shocking capacitor 632 may be charged to an initial voltage of 475 V, which drops over the course of a positive phase of a single biphasic MV shock to between 375 V-400 V, and further drops during a negative phase of the same single biphasic MV shock to between 325 V-375 V. However, the control signal 700 may lengthen the duty cycle of successive control pulses over the positive and negative phases to provide an effective stimulation waveform that exhibits a substantially constant maximum voltage of 100V-200 V during the positive phase and 100V-200 V during the negative phase of the single biphasic MV shock. For example, with reference to FIG. 6, during delivery of a single phase therapy and/or multiphase therapy, the feedback circuit 634 may continuously sense the voltage level on the storage capacitor(s) 632 and provide the sensed voltage to the control circuitry 636 and comparison circuitry 626. The control circuitry 636 and/or comparison circuitry 626 adjust a duty cycle of control pulses within the control signals delivered to the switching devices 642-648 to maintain an effective waveform shape within the therapy. Hence, the feedback circuitry 634 senses voltage drops across the storage capacitor(s) 632 and the control circuitry 636 and/or comparison circuitry 626 compensates for the drop in voltage by extending the duty cycle of individual control pulses (e.g., increasing the duty cycle from 10%, to 20%, 30% and upward toward 100%).

In the embodiment of FIG. 7, the control pulses 702-705 manage generation of the positive phase segment of a biphasic MV shock, while the control pulses 706-709 manage generation of the negative phase segment of the biphasic MV shock.

The control pulses 702-709 are turned on and off with a desired duty cycle. The duty cycles of successive pulses differ from one another. For example, the duty cycles of earlier control pulses 702, 703 are relatively small (e.g., 10-20%), as compared to the duty cycles of the later control pulses 704, 705. The duty cycle of successive control pulses is adjusted based on various criteria. For example, the duty cycle may be adjusted to achieve a desired shape in the effective stimulation waveform. As one example, control pulses proximate a leading-edge of a positive or negative phase of the stimulation shock may have a relatively low duty cycle (e.g., 10-20%), while the control pulses proximate the trailing edge of the positive or negative segment of the stimulation shock have a longer duty cycle (e.g., 30-80%).

Additionally or alternatively, the duty cycle may be adjusted as energy is drawn from the shocking capacitor or bank of shocking capacitors, in order to compensate for a drop in the voltage of the capacitor(s). For example, as the shocking capacitor(s) discharge energy during one or more MV shocks, the control circuit may lengthen the duty cycle such that successive control pulses remain in an ON or high state for progressively longer percentages of the period. By progressively extending the duty cycle, as the peak voltage of the shocking capacitor(s) drops, embodiments herein maintain a relatively constant energy discharge from the capacitor(s) during a single period. To continue the foregoing example, the energy delivered in response to a single control pulse is based on a pulse width of the control pulse and based on the charge of the capacitor(s) (typically defined in terms of voltage).

The control pulse widths for each phase of the shock start at the lower values and increment to the maximum value by the end of the phase of the shock. Smaller pulse widths produce lower “effective” voltages and longer pulse widths produce higher “effective” voltages. The example of FIG. 7 will produce an effective waveform that includes an ascending ramp shaped leading edge profile as the shock voltage moves from a 0V to a voltage below the stored voltage on the shocking capacitor(s). The capacitor(s) will lose some of an initial full charge during the ramp up process and thus the effective waveform maximum voltage will not necessarily reach the initial voltage stored on the capacitor(s). Additionally or alternatively, the capacitor(s) may be charged to a significantly higher voltage than the output voltage of the shock, in which case, a constant low duty-cycle pulse width may be utilized for the control pulses to produce a constant effective voltage (e.g., similar to the square waveforms in FIG. 5). For example, a capacitor charged to 475V can produce two, three or more shocks at 100V each.

FIG. 8 illustrates an example of an effective stimulation waveform 802 experienced by a heart during delivery of a single biphasic MV shock in accordance with an embodiment herein. The waveform 802 may be delivered as a single stand-alone biphasic shock, or a series of two or more biphasic shocks. The effective stimulation waveform 802 includes a positive phase segment 804 and a negative phase segment 806. The positive phase segment 804 comprises a leading-edge profile 808, an intermediate profile 808 (illustrated as a steady-state plateau segment) and a trailing edge profile 810. The negative phase segment 806 comprises a leading-edge profile 811, an intermediate profile 812 (illustrated as a steady-state plateau segment) and a trailing edge profile 813. The leading edge profile 808 extends from a reference zero voltage to a positive peak voltage along an ascending ramp, wherein the slope of the ramp progressively increases from a horizontal to a vertical slope. The rate of increase in the slope is controlled and determined by the duty cycle of the control pulses delivered to the switching circuit by the control circuit. The leading-edge profile 811 extends from a reference zero voltage to a negative peak voltage along a descending ramp, where in the slope of the ramp progressively decreases from a vertical slope to a horizontal slope. The rate of change in the slope is controlled and determined by the duty cycle of the control pulses. The leading-edge profile 808 and steady-state profile 809 intersect at a transition point that may be shaped in various manners. In the example of FIG. 8, the transition point is shown to be generally a square corner. Additionally or alternatively, the transition point may be rounded to a smoother transition, such as to form a curve or other bend.

The shock waveform of FIG. 8 differs from shock waveforms generated by conventional systems, both in shape and in maximum voltage. Conventional systems generate a waveform that exhibit a leading-edge that extends vertically substantially straight up or at an extremely steep slope to jump from a zero voltage to a maximum peak voltage of 800 V-900 V in an extremely short period of time. The extremely steep slope on the leading edge of a conventional shock potentially damages tissue near the electrode and is inefficient as the surrounding myocardial cells are unable to effectively process the large amount of energy/high-voltage immediately introduced.

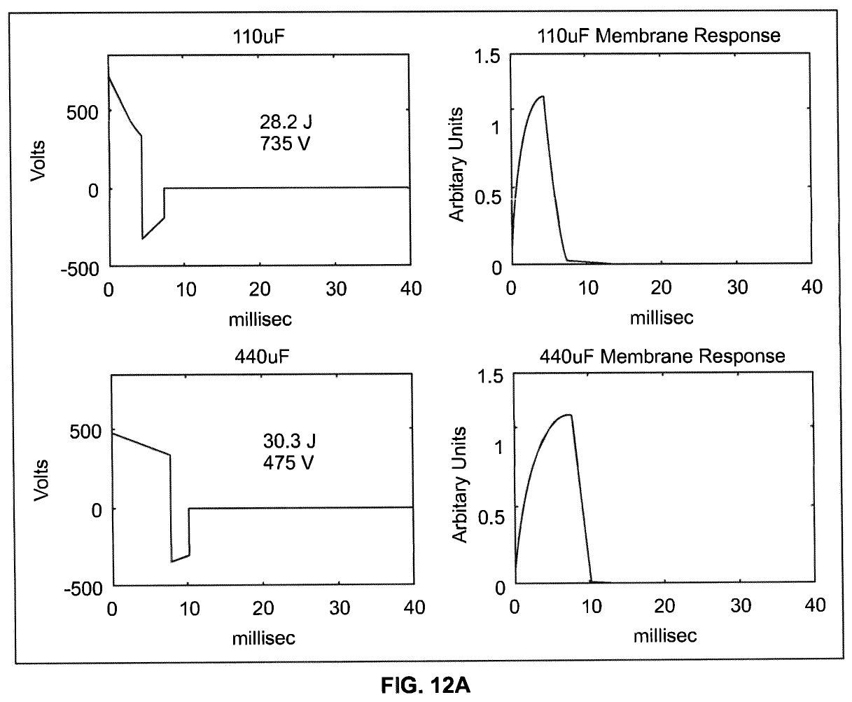

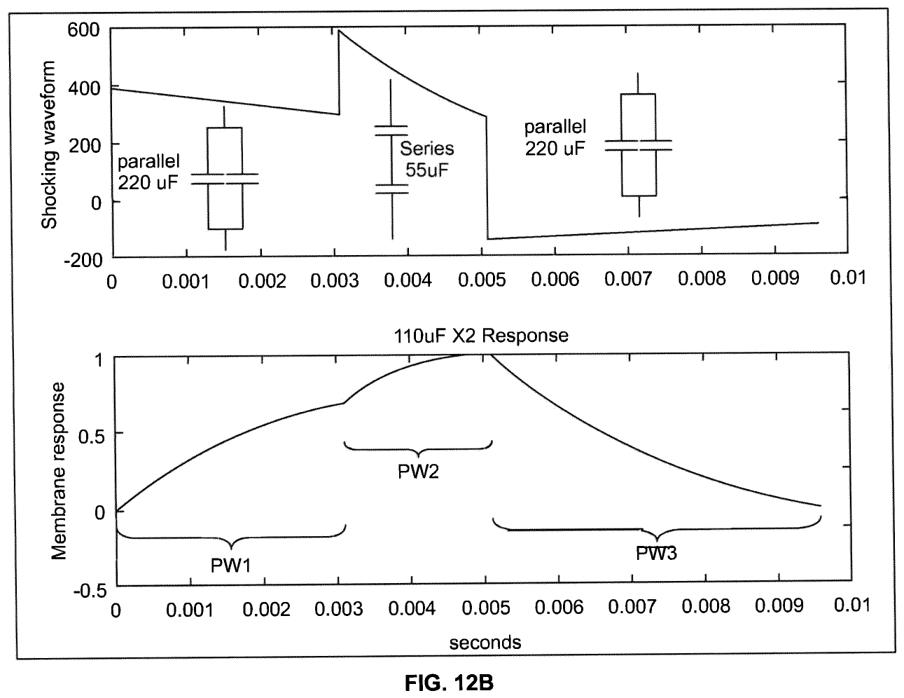

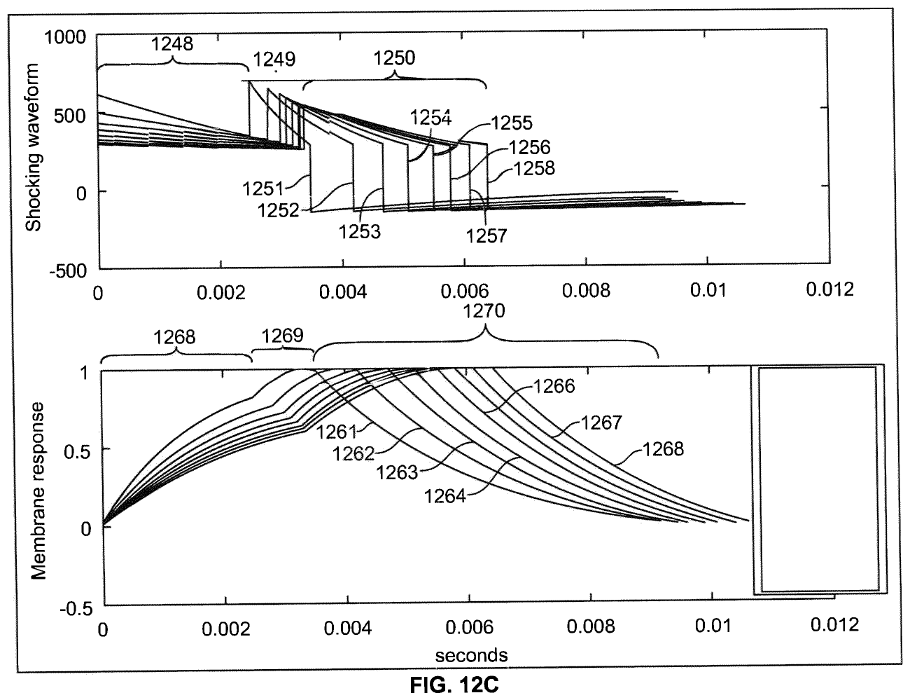

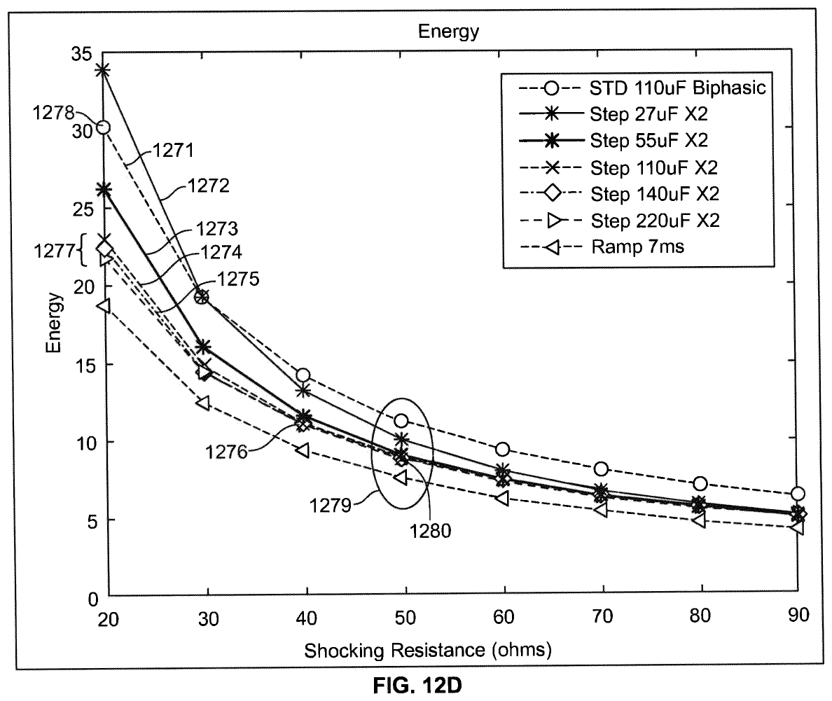

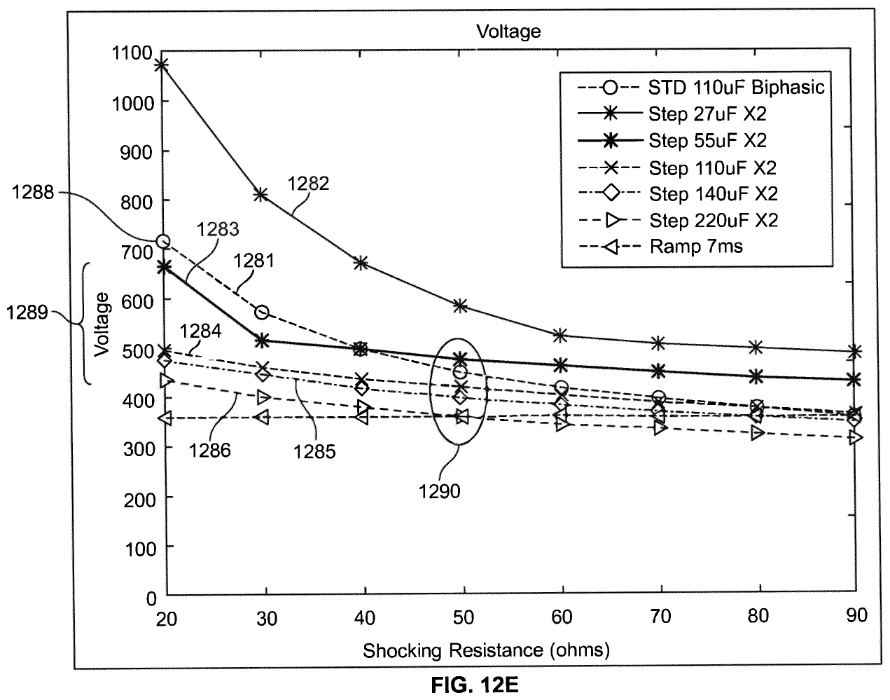

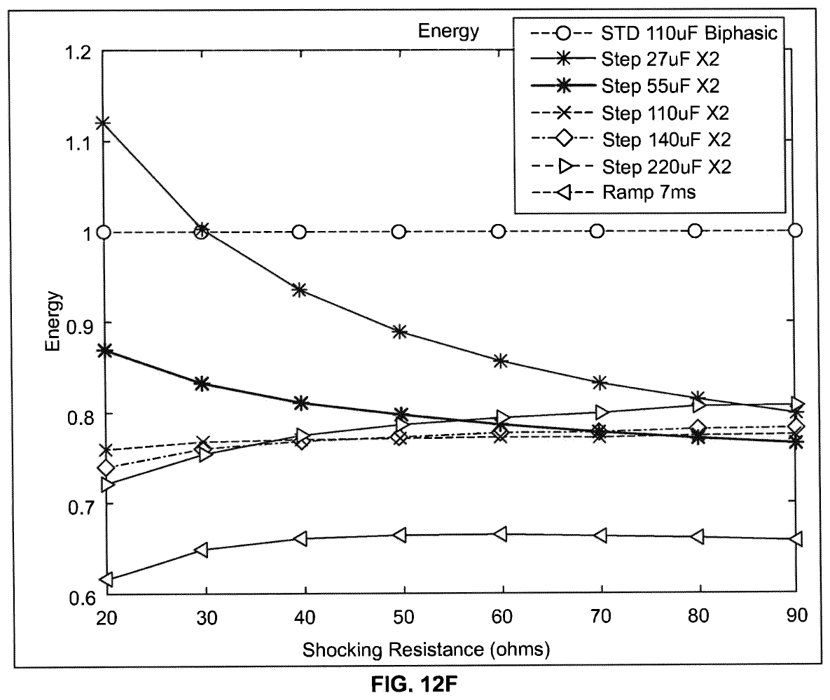

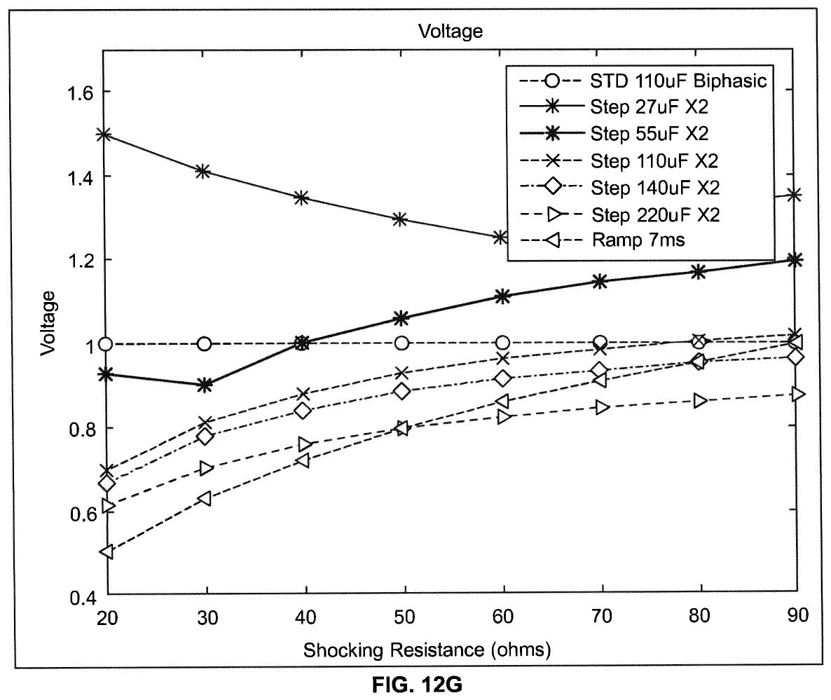

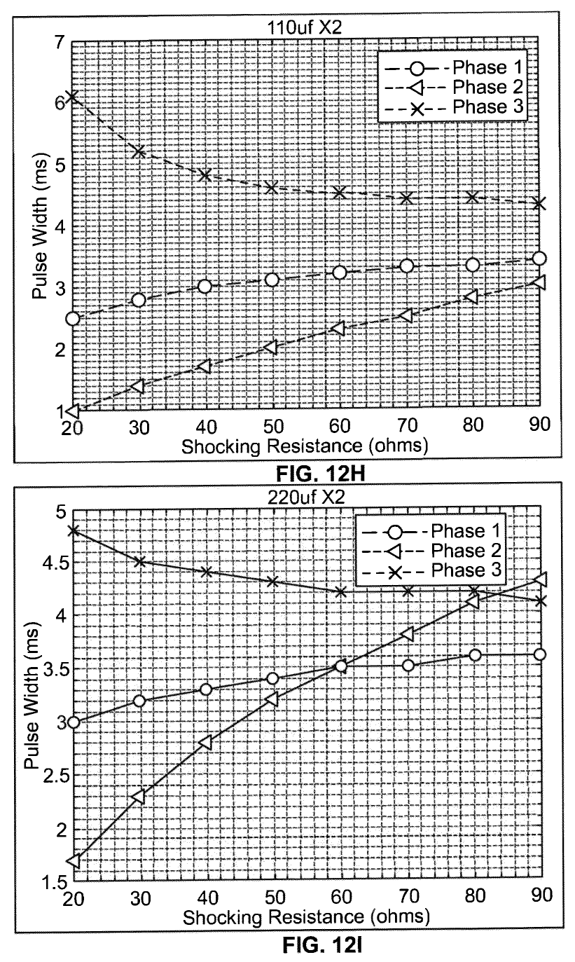

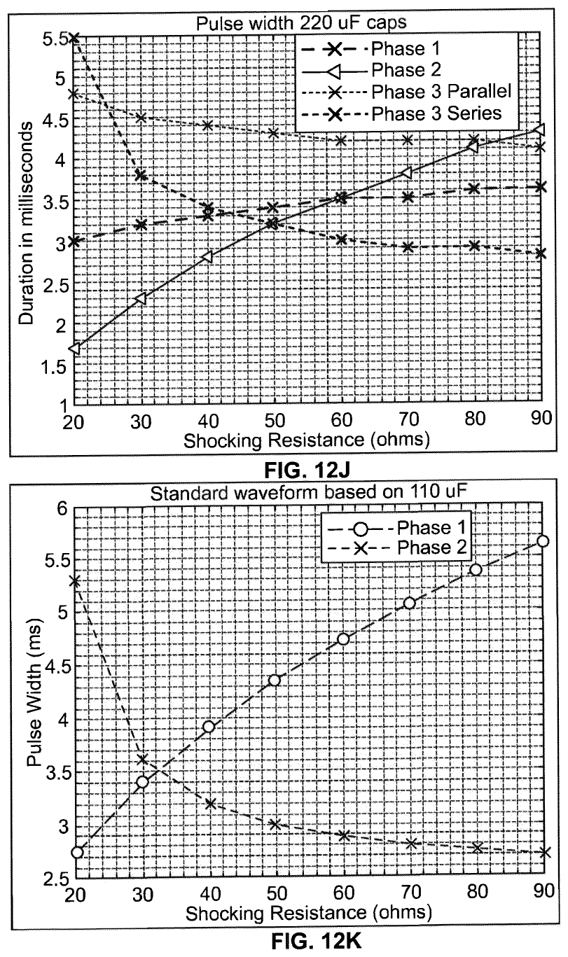

In accordance with embodiments herein, the leading edge profiles 808, 811 is curved along an ascending ramp with a progressively changing (e.g., increasing or decreasing) slope to move from the reference zero voltage to the positive or negative peak medium voltages over a desired ramp up period of time, to form a desired shape for the leading-edge profiles 808, 811. By wave of example, the leading edge profiles 808, 811 may follow a ramp having an exponentially increasing or decreasing shape, bell shape, or more generally a curve defined by a second order, third order or higher order polynomial function. The rate and shape at which the voltage moves from the reference zero voltage to the positive/negative peak voltages may be varied to avoid damage to tissue in regions immediately surrounding the electrodes delivering the shock. Also, the rate and shape at which the voltage moves from the reference zero voltage to the positive/negative peak voltages may be varied to achieve a desired level of efficiency in which the myocardial cells process and fully utilize the energy/voltage introduced. Thus, by utilizing an ascending ramp during at least the leading-edge profiles 808, 811, the effective stimulation waveform 802 is more efficient, as compared to a conventional shocking waveform. Optionally, the positive phase segment 804 may include multiple ascending ramp segments that are stepped with respect to one another. For example, the positive phase segment 804 may include first and second waveform segments as described below in connection with FIGS. 12A-12J. Optionally, the negative phase segment 806 may include an ascending ramp segment as described below in connection with FIGS. 12A-12J.

Once the voltage transitions from the leading-edge segment to the intermediate profiles 809, 812, the voltage reaches a positive or negative peak level. The peak voltage may be maintained for all or a portion of the profiles 809, 812, such as a generally steady-state voltage across the profiles 809, 811. Thereafter, the effective stimulation waveform 802 transitions from the positive or negative peak level to the reference zero voltage along a desired curve, corresponding to the trailing edge profiles 810, 813. In the example of FIG. 8, the trailing edge profiles 810, 813 are curved, such as to follow an exponential curve, bell curve or other polynomial curve.

Optionally, the trailing edge profiles 810, 813 may have alternative shapes that differ from one another. In some embodiments, the leading and trailing edge profiles 808, 810 and 811, 813 may exhibit generally the same shape, while in other embodiments the leading-edge profiles 808, 811 may have a shape that differs from the shape of the trailing edge profiles 810, 813. In a further alternative embodiment, the shape of the leading-edge profile 808 may differ from the shape of the leading-edge profile 811, and the shape of the trailing edge profile 810 may differ from the shape of the trailing edge profile 813. In the present example, the profiles 809, 812 correspond to positive and negative peak voltages that are substantially the same, although it is recognized that in alternative embodiments the peak voltage of the negative profile 812 may be less or greater than the peak voltage of the positive profile 809. As a further example, the shapes of the positive and negative profiles 809, 812 may not follow a generally constant plateau voltage. Instead, the shapes of the profiles 809, 812 may exhibit an exponentially decaying voltage across the profile 809, 812 (e.g., similar to the exponential decaying shape of the segments in FIG. 4, and/or any other desired shape).

Optionally, the control circuit of FIG. 6 may be utilized to define an effective waveform having any desired shape.

### Switching Between Series and Parallel Capacitor Configurations

Convention implantable defibrillators house two or more defibrillation capacitors that are permanently attached to one another in series. Typically, each of the capacitors has a capacitance of 220 μF and are charged to 450-500V, thereby resulting in a series capacitance of 110 μF with a maximum voltage of 900V-1000V. A switching circuit connects the series capacitors to a shocking electrode combination to deliver a first (e.g., positive) pulse of a single biphasic shock. After a period of time, corresponding to the pulse width of the positive pulse of the biphasic single shock, the switching circuit changes a switch configuration to change a polarity of the connection between the series capacitors and the shocking electrodes. The series capacitors then delivery a second (e.g., negative) pulse of the biphasic shock, still with an effective capacitance of 110 μF. The leading peak voltage of the negative pulse of the biphasic shock has an amplitude corresponding to the remaining charge on the series capacitors after delivering the positive pulse of the biphasic shock. The positive and negative pulses exhibit an exponentially decaying voltage where the shape of the exponential curve is defined in part based on the effective capacitance (e.g., 110 μF) and effective voltage (e.g., 1000V for 1^{st} pulse).

However, this convention series capacitor configuration experiences certain limitations. For example, the biphasic shock with the foregoing shape requires a certain level of energy to successfully defibrillate a heart, namely the DFT. The voltage for the DFT is relatively high which causes a high initial peak current density and high myocardial electric field intensity at the shocking electrodes.

In accordance with embodiments herein, methods and systems are described that reduce the voltage required for the DFT, as well as reduce the shock peak current density and reduce the myocardial electric field intensity. Among other things, reducing the shock peak current density and electric field intensity prevents/reduces tissue damage in the regions immediately adjacent the shocking electrodes.

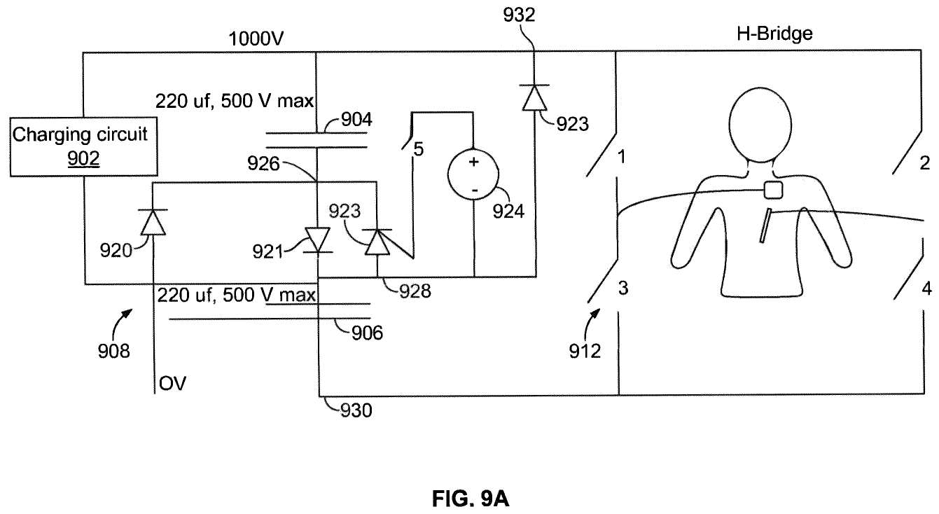

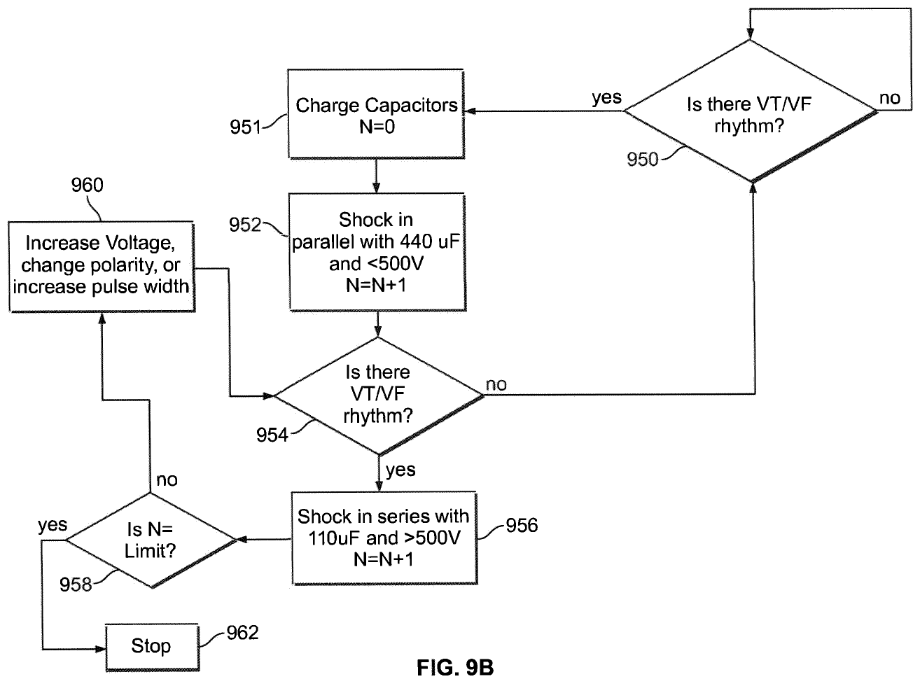

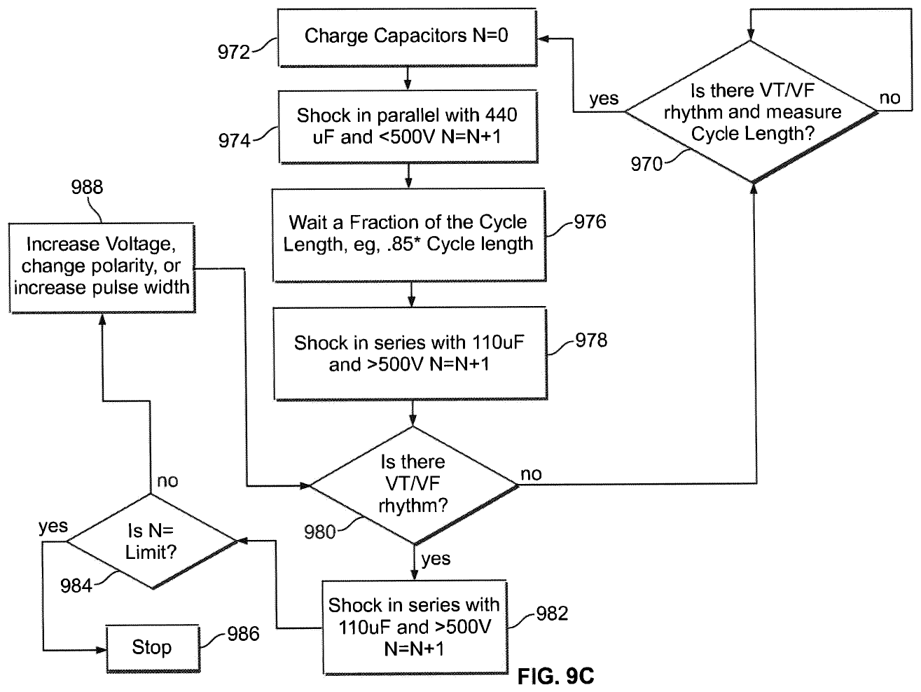

FIG. 9A illustrates a schematic diagram of at least a portion of a circuit utilized in an IMD to manage delivery of one or more biphasic shocks in accordance with embodiments herein. The circuit of FIG. 9A may deliver one or more shocks as part of a phase I therapy in connection with the multi-phase therapies described herein. Alternatively, the circuit of FIG. 9A may delivery the one or more shocks as the only defibrillation therapy, namely a phase I therapy only, without the IMD delivering any phase II or III therapy. The IMD includes a charging circuit 902 that is connected to a reconfigurable capacitor bank that includes two capacitors 904, 906 that are connected through a parallel/series reconfiguration (PSR) circuit 908. Optionally, the PSR circuit (and/or other portions of the charging and shocking circuit) may be formed in accordance with the methods and systems described in U.S. Pat. No. 6,233,483, titled “SYSTEM AND METHOD FOR GENERATING A HIGH EFFICIENCY BIPHASIC DEFIBRILLATION WAVEFORM FOR USE IN AN IMPLANTABLE CARDIOVERTER/DEFIBRILLATOR (ICD)”, issuing May 15, 2001, and/or U.S. Pat. No. 6,714,818, titled “SYSTEM AND METHOD FOR GENERATING AN OPTIMAL THREE-STEP DEFIBRILLATION WAVEFORM FOR USE IN AN IMPLANTABLE CARDIOVERTER/DEFIBRILLATOR (ICD)”, issuing Mar. 30, 2004 (both attached at appendix A), the complete subject matter which are expressly incorporated herein by reference in their entirety.

The PSR circuit 908 reconfigures the connections between the capacitors 904, 906 between a parallel configuration and a series configuration as described herein. The reconfigurable capacitor bank is connected through a switching circuit 910 to shocking electrodes.

During first (e.g., positive) and second (e.g., negative) pulses of at least a first biphasic shock, the PSR circuit 908 connects and configures the capacitors 904, 906 in a parallel configuration (also referred to as a parallel biphasic shock). Optionally, the PSR circuit 908 may maintain the parallel configuration for more than one biphasic shock, such as for a series of MV shocks as described above in connection with various phase I therapies.

By way of example, the capacitors 904, 906 may have capacitances of 220 μF and be charged to 450V-500V. Thus, when connected in parallel the effective capacitance is 440 μF with a maximum leading voltage of 450V-500V. Thus, when connected in series the effective capacitance is 110 μF with a maximum leading voltage of the sum of the voltage on each capacitor 904, 906. When the capacitors 904, 906 are fully charged, the maximum leading voltage is 900V-1000V. In accordance with the foregoing, the PSR circuit 908, reconfigures the capacitor bank to have first and second effective capacitances during first and second biphasic shocks, respectively. The first and second biphasic shocks similarly exhibit first and second exponential decays, respectively. The parallel configuration of the capacitors induces less damage into the surrounding tissue, while the series configuration (when delivering high voltage/energy) may be more effective. Thus, switching between parallel and series configurations affords the benefits of both less tissue damage while remaining effective.

A switching circuit 912 connects the parallel capacitors 904, 906 to a shocking electrode combination to deliver a first (e.g., positive) pulse of a single biphasic shock (e.g., closing switches 1 and 3). After a period of time, corresponding to the pulse width of the positive pulse of the biphasic single shock, the switching circuit 912 changes a switch configuration to change a polarity of the connection between the parallel capacitors and the shocking electrodes (e.g., opening switches 1 and 3, and closing switches 2 and 4). The parallel capacitors then delivery a second (e.g., negative) pulse of the biphasic shock, still with an effective capacitance of 440 μF. The leading peak voltage of the negative pulse of the biphasic shock has an amplitude corresponding a remaining charge on the parallel configuration of capacitors after delivering the positive pulse of the biphasic shock. The positive and negative pulses exhibit an exponentially decaying voltage where the shape of the exponential curve is defined in part based on the effective capacitance (e.g., 440 μF) and effective voltage (e.g., 450V for 1^{st} pulse).

Next, during first (e.g., positive) and second (e.g., negative) pulses of a second or later biphasic shock, the PSR circuit 908 connects and configures the capacitors 904, 908 in a series configuration (also referred to as a series biphasic shock). The switching circuit 912 connects the series capacitors 904, 906 to a shocking electrode combination to deliver a first (e.g., positive) pulse of a single biphasic shock (e.g., closing switches 1 and 3). After a period of time, corresponding to the pulse width of the positive pulse of the biphasic single shock, the switching circuit 912 changes a switch configuration to change a polarity of the connection between the series capacitors and the shocking electrodes (e.g., opening switches 1 and 3, and closing switches 2 and 4). The parallel configuration of capacitors then delivery a second (e.g., negative) pulse of the biphasic shock, still with an effective capacitance of 110 μF. The leading peak voltage of the negative pulse of the biphasic shock has an amplitude corresponding a remaining charge on the series capacitors after delivering the positive pulse of the biphasic shock. The positive and negative pulses exhibit an exponentially decaying voltage where the shape of the exponential curve is defined in part based on the effective capacitance (e.g., 110 μF) and effective voltage (e.g., 900V for 1^{st} pulse).

Optionally, the PSR circuit 908 may maintain the series configuration for one biphasic shock or more than one biphasic shock. By way of example, the IMD may direct delivery of one or more MV shocks using the parallel capacitor configuration, and then sense and analyze additional cardiac events. When the additional cardiac events indicate that the VF episode has terminated, the IMD may cancel the series biphasic shock. When the additional cardiac events indicate that the VF episode has not terminated, the IMD directs the PSR circuit 908 to change to the series configuration and deliver one or more series biphasic shocks.

The PSR circuit 908 includes diodes 920-923 that switch between closed and open states. A power source 924 is coupled to diode 922 through switch 5.

When the switch 5 is open (OFF), the SCR diode 922 is turned OFF and the nodes 926, 928 are disconnected from one another. When the nodes 926, 928 become disconnected from one another, the diode 920 becomes forward bias to connect the negative terminal of the capacitor 904 to node 930 (e.g., ground). When the nodes 926, 928 become disconnected from one another, the diode 921 enters a reverse bias state and forms an open circuit/state, while the diode 932 becomes forward bias. When the diode 932 is forward bias, it forms a closed circuit/state, thereby connecting positive terminal of the capacitor 906 to node 932. In the foregoing configuration, the positive terminals of the capacitors 904, 906 are connected to a common node 932 (high voltage), and the negative terminals of the capacitors 904, 906 are connected to a common node 930 (ground), thereby providing an effective capacitance of 440 F and peak voltage corresponding to the voltage across the parallel capacitors 904, 906 (e.g., 500V). When the SCR diode 922 is OFF, the parallel capacitors a medium drive current in parallel providing a MV shock with a higher relative effective capacitance (e.g., 440 μF).

When the switch 5 is closed (ON), the diode 922 (e.g., an SCR) is turned on and connects nodes 926 and 928 to one another, thereby connecting and configuring the capacitors 904, 906 in series, thereby providing an effective capacitance of 110 F and peak voltage corresponding to the voltage across the series capacitors 904, 906 (e.g., 1000V). When the SCR diode 922 is ON, the series capacitors drive a high current in series providing a HV shock with a lower relative effective capacitance (e.g., 110 μF).

During operation, once a VF episode is identified, the PSR circuit would connect the capacitors in parallel and charge the capacitors an initial voltage (e.g., 500V). The parallel biphasic shock is delivered having a relatively flat exponential decay in each of the positive and negative pulses of the biphasic shock. The exponential decay of the parallel biphasic shock is flat relative to the exponential decay of a series biphasic shock. Next, the IMD would sense new cardiac events and determine if the VF episode has terminated. If so, the process ends. If the VF episode continues, the capacitors would be recharged and the PSR circuit would reconfigure the capacitor bank to have the series configuration described herein. Next, a series biphasic shock would be delivered.

FIG. 9B illustrates a flowchart that may be implemented in connection with embodiments herein to deliver more than one shock while switching between capacitor series and parallel configurations. Beginning at 950, the IMD monitors cardiac activity, analyzes cardiac activity signals to identify whether the patient is experiencing VT/VF arrhythmia. When a VT/VF arrhythmia is identified, flow moves to 951. At 951, the one or more processors of the IMD initiate a charge capacitor instruction and sets a count of the number of shocks delivered N=0. At 952, the capacitor bank configures the capacitors in a parallel configuration, such that two 220 μF capacitors yield an effective capacitance of 440 μF. The capacitors are fully charged to a desired voltage level (e.g., less than 500 V) and a shock is delivered. The shock counter N is incremented. At 954, the one or more processors determine whether the patient is still exhibiting a VT or VF arrhythmia. If the patient is no longer experiencing a VT/VF arrhythmia, the processor determined that the arrhythmia has been terminated and flow returns to 950.

At 954, when the one or more processors determined that the VT/VF arrhythmia has not been terminated, flow moves to 956. At 956, the one or more processors reconfigure the capacitors to a series configuration, thereby resulting in an effective capacitance of approximately 110 μF. At 956, when the capacitors are connected in series, the resulting peak voltage will be greater than 500 V. Optionally, the charge on the capacitors at 956 may simply represent the residual charge remaining on the capacitors after delivering the shock at 952. Alternatively, at 956, the capacitors may be recharged, with each capacitor being charged to a level less than 500 V (but with the series configuration having a resulting voltage greater than 500 V). At 956, the shock count is incremented N=N+1.

Next flow moves to 958 where the one or more processors determine whether the shock count has reached a limit. When the shock count reaches the limit, flow moves to 962 and the process stops. Alternatively, when the shock count has not yet reached the limit, flow moves from 958 to 960. At 960, the one or more processors may change one or more of the shock parameters, such as increasing the voltage to which the capacitors are charged, changing the polarity, increasing the pulse width and the like. Optionally, in accordance with new and unique aspects herein, the shape of the defibrillation waveform may be modified, such as to resemble one or more of the waveforms described in connection with FIG. 8 and/or FIGS. 12A-12 J. Optionally, the operations at 952 may be repeated more than once to deliver more than one medium voltage high capacitance shock.

FIG. 9C illustrates a flowchart that may be implemented in connection with embodiments herein to deliver more than one shock while switching between capacitor series and parallel configurations. Beginning at 970, the IMD monitors cardiac activity, analyzes cardiac activity signals to identify whether the patient is experiencing VT/VF arrhythmia. When a VT/VF arrhythmia is identified, flow moves to 972. At 972, the one or more processors of the IMD initiate a charge capacitor instruction and sets a count of the number of shocks delivered N=0. At 974, the capacitor bank configures the capacitors in a parallel configuration, such that two 220 μF capacitors yield an effective capacitance of 440 μF. The capacitors are fully charged to a desired voltage level (e.g., less than 500 V) and a shock is delivered. The shock counter N is incremented. At 976, the processors wait a predetermined period of time, such as a fraction of a cycle length (e.g., 85% of a cycle length). Thereafter, at 978, the one or more processors reconfigure the capacitors into a series configuration and deliver a shock having an effective capacitance of 110 μF and an initial voltage of greater than 500 V. Optionally, the charge on the capacitors at 978 may simply represent the residual charge remaining on the capacitors after delivering the shock at 976. Alternatively, at 978, the capacitors may be recharged, with each capacitor being charged to a level less than 500 V (but with the series configuration having a resulting voltage greater than 500 V). At 978, the shock count is incremented N=N+1.

At 980, the one or more processors determine whether the patient is still exhibiting a VT or VF arrhythmia. If the patient is no longer experiencing a VT/VF arrhythmia, the processor determined that the arrhythmia has been terminated and flow returns to 970. At 980, when the one or more processors determined that the VT/VF arrhythmia has not been terminated, flow moves to 982. At 982, the one or more processors retain the capacitors in the series configuration, thereby resulting in an effective capacitance of approximately 110 μF. At 982, when the capacitors are connected in series, the resulting peak voltage will be greater than 500 V. Optionally, the charge on the capacitors at 982 may simply represent the residual charge remaining on the capacitors after delivering the shock at 978. Alternatively, at 982, the capacitors may be recharged, with each capacitor being charged to a level less than 500 V (but with the series configuration having a resulting voltage greater than 500 V). At 982, the shock count is incremented N=N+1.

Next flow moves to 984 where the one or more processors determine whether the shock count has reached a limit. When the shock count reaches the limit, flow moves to 986 and the process stops. Alternatively, when the shock count has not yet reached the limit, flow moves from 984 to 988. At 988, the one or more processors may change one or more of the shock parameters, such as increasing the voltage to which the capacitors are charged, changing the polarity, increasing the pulse width and the like. Optionally, in accordance with new and unique aspects herein, the shape of the defibrillation waveform may be modified, such as to resemble one or more of the waveforms described in connection with FIG. 8 and/or FIGS. 12A-12 J. Optionally, the operations at 974 may be repeated more than once to deliver more than one medium voltage high capacitance shock.

### Multiple MV Shocks Plus HV Shock

In accordance with the foregoing description of FIGS. 9A-11, a single parallel biphasic MV shock may be delivered before switching the capacitor bank configuration to deliver a single series biphasic HV shock, as part of a phase I therapy alone or followed by a phase II therapy and/or phase III therapy. Additionally or alternatively, two or more parallel biphasic MV shocks may be delivered before switching the capacitor bank configuration to deliver one or more series biphasic HV shocks, as a phase I therapy alone or followed by phase II and/or III therapies. The process may deliver one or more shocks as part of a phase I therapy in connection with the multi-phase therapies described herein. Alternatively, the process may delivery the one or more shocks as the only defibrillation therapy, namely a phase I therapy only, without the IMD delivering any phase II or III therapy. More specifically, as one example process, once cardiac events are sensed and analyzed to detect a VF episode, the capacitor bank may be configured in a parallel configuration and charge to a maximum voltage (e.g., 500 V). In the above example, when each individual capacitor has a capacitance of 220 μF, connecting the capacitors in parallel yields an effective capacitance of 440 μF. Once fully charged, the process delivers an initial parallel biphasic shock. In accordance with embodiments herein, when the phase I therapy includes more than one MV shock, after the first MV shock, the process may wait a predetermined shock to shock interval (e.g., 50 to 350 ms). Once the predetermined shock to shock interval times out, the process delivers a second parallel biphasic MV shock. By way of example, the predetermined shock to shock interval may be defined automatically by the IMD during operation based on a cycle length of the arrhythmia. For example, during the arrhythmia detection process, the IMD may identify the cycle length of the arrhythmia and assign the shock to shock interval to correspond to a select percentage of the arrhythmia cycle length. The first and second MV shocks are delivered following, and based upon, a single common charge of the capacitor bank. The capacitor bank is not recharged between the first and second MV shocks.

Following delivery of the second MV shock, the charging circuit recharges the capacitor bank to the maximum initial voltage (e.g., 500 V) and the PSR circuit reconfigures the capacitors in a series configuration. In the above example, when each individual capacitor has a capacitance of 220 μF, connecting the capacitors in series yields an effective capacitance of 110 μF. Once the capacitor bank is recharged to its initial charge (or an alternative predetermined charge for delivery of and HV shock), the IMD delivers the series biphasic HV shock.

The foregoing process for delivering multiple in the shocks followed by an HV shock yields certain advantages. For example, empirical testing has shown that 2 MV shocks delivered together closely may provide a more effective overall phase I therapy. The multiple MV shocks in the phase I therapy should have a similar effect on myocardial tissue. To yield a similar effect on the myocardial tissue, at least some embodiments maintain a substantially common initial voltage (or within a predetermined range) for each positive and each negative pulse within the first and second MV shocks. Additionally or alternatively, the initial voltage is of the positive and negative pulses in the first and second MV shocks may be maintained within a desired voltage range of one another. As one nonlimiting example, the initial voltage of the positive pulse the second MV shock may be within 1%-20% of the initial voltage of the positive pulse in the first MV shock. As one nonlimiting example, the initial voltage of the positive pulse the second MV shock may be within 1%-5% of the initial voltage of the positive pulse in the first MV shock. As one nonlimiting example, the initial voltage of the positive pulse the second MV shock may be within 5%-10% of the initial voltage of the positive pulse in the first MV shock. Embodiments herein are able to maintain an initial voltage of the pulses of the second MV shock within a desired range of the initial voltage is of the first MV shock by providing a very small droop or drop in the exponential decay of the waveform shape across than individual poles of a biphasic shock. The droop or drop exponential decay is defined in part by the effective capacitance of the capacitor bank. By way of example, the capacitor bank, having an effective capacitance of 440 μF, defines an exponential decay with relatively little droop. For example, the droop between the initial and final voltages of an individual pulse within a shock may be between 2% and 60%, more preferably between 10% and 50%, and more preferably, less than 25%. By maintaining substantially similar voltage profiles (e.g., initial and final voltages) across the positive and negative pulses of the first and second MV shocks, embodiments herein you will a substantially similar effect upon the membrane potential by both of the MV shocks.

The parallel biphasic shock having a relatively high capacitance is more effective than a biphasic shock having a similar leading voltage but lower effective capacitance. For example, a 500V, 440 μF shock would exhibit lower DFTs as compared to a 500V, 110 μF shock, all other factors being equal.

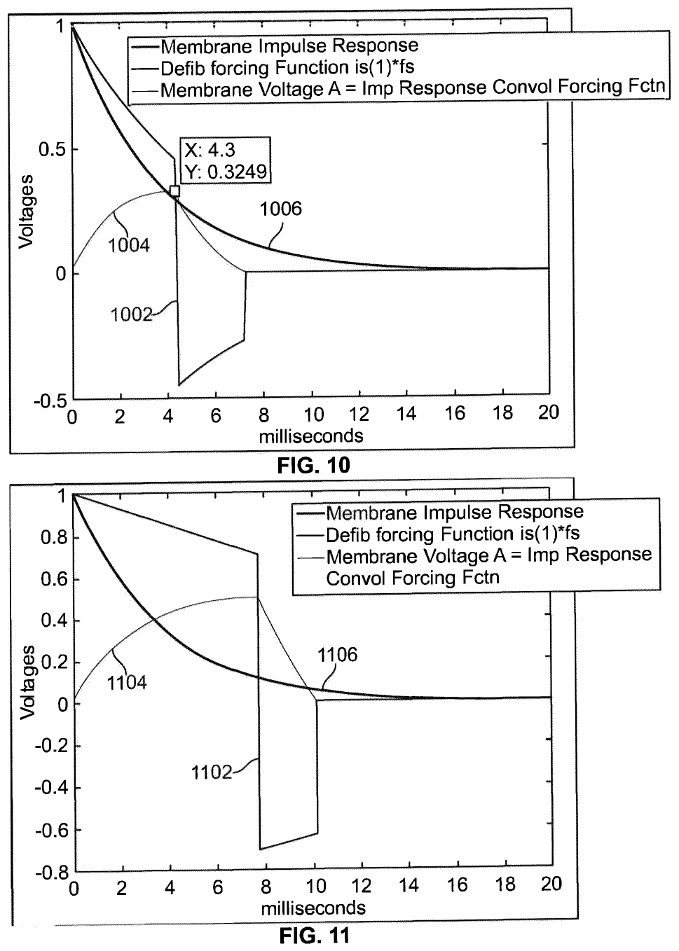

FIGS. 10 and 11 illustrate three plots, namely a membrane impulse response, a defibrillation forcing function and a membrane voltage A=Impulse response convolved with the forcing function. It is believed that a lower voltage pulse may be of significant benefit because the tissue is exposed to lower peak currents.

FIGS. 10 and 11 illustrate theoretical responses, as normalized on a scale of −0.5-1.0 (vertical axis) and over a 20 ms time interval (horizontal axis). The myocardial cells exhibit behavior that can be modeled as a parallel RC network having a predetermined time constant. For example, the myocardial cells can be modeled as a parallel RC network having a time constant of approximately 3.5 ms. During delivery of a defibrillation shock, the IMD acts to produce a forcing function that drives current into the parallel RC network model of the myocardial cells. The voltage and current delivered by the IMD in turn produces the forcing function. The resulting voltage experienced by the cellular membranes can be calculated as the convolution of the membrane impulse response and the defibrillation forcing function.

FIG. 10 illustrates a theoretical response of the cell membrane voltage when a unitary biphasic defibrillation shock 1002 is delivered from a capacitor bank exhibiting a 110 μF effective capacitance. The membrane impulse response is illustrated at 1004 and the convolution of the membrane impulse response 1004 and the defibrillation forcing function 1002 are illustrated as the resulting voltage experienced by the cellular membranes at 1006. The membrane potential rises to 0.325 and with the negative phase of the shock the membrane potential is precisely driven to zero volts. This is believed to reduce the probability of re-initiation of the arrhythmia. The phase 1 and phase 2 durations may be optimized according to the process described in the publication to Kroll et al, “ICD Waveform Optimization: A Randomized, Prospective, Pair-Sampled Multicenter Study” Pacing and Clinical Electrophysiology, 23:1992-1995. doi:10.1111/j.1540-8159.2000.tb07070.x PACE 2000; 23:[Pt.11]:1992-1995, the complete subject matter of which is expressly incorporated herein by reference in its entirety.

FIG. 11 shows the response to the same amplitude pulse as in FIG. 10 but derived from a 440 μF effective capacitance. The phase 1 and phase 2 pulse widths are optimized by the same methods described by Kroll et al. FIG. 11 illustrates a theoretical response of the cell membrane voltage when a unitary biphasic defibrillation shock 1102 is delivered from a capacitor bank exhibiting a 440 μF effective capacitance. The membrane impulse response is illustrated at 1104 and the convolution of the membrane impulse response 1104 and the defibrillation forcing function 1102 are illustrated as the resulting voltage potential experienced by the cellular membranes at 1106. The membrane potential rises to 0.325 and with the negative phase of the shock the membrane potential is precisely driven to zero volts. As exhibited in FIG. 11, the higher effective capacitance of the capacitor bank, namely 440 μF, causes the 440 μF shock waveform to raise the membrane potential to a normalized value of 0.503, which is 55% greater than the membrane potential of the 110 μF shock waveform in FIG. 10. The foregoing analysis can be extrapolated to an IMD that would utilize a 110 μF effective capacitance, thereby indicating that that a 500 V shock delivered by a 440 μF capacitor is equivalent in voltage to a 773 V shock delivered by a 110 μF capacitor.

### Ascending Stepped Waveform

In accordance with new and unique aspects herein, it is been found that lower voltage defibrillation pulses provide a same membrane response as conventional higher voltage defibrillation pulses, but with only a modest increase in energy. FIG. 12A illustrates four panels, with the upper left illustrating an example biphasic shock waveform (e.g., having an initial voltage of 735 V and an energy content of 28.2 J) delivered from a capacitor bank having an effective capacitance of 110 μF. The upper right panel illustrates an example of a membrane response experienced in response to the shock waveform in the upper left panel. The lower left panel illustrates an example biphasic shock waveform (e.g., having an initial voltage of 475 V and an energy content of 30.3 J) delivered from a capacitor bank having an effective capacitance of 440 μF. The lower right panel illustrates an example of a membrane response experienced in response to the shock waveform in the lower left panel.

FIG. 12B presents an example model of an ascending stepped waveform formed in accordance with embodiments herein, along with a model of the membrane response thereto. The upper panel illustrates the shock ascending stepped waveform along with examples of capacitor configurations that may be utilized to form the shock waveform. The upper panel illustrates capacitor instantaneous discharge voltage along the vertical axis and time along the horizontal axis. The ascending stepped waveform includes first and second waveform segments PW1 and PW2 that are delivered during a first (e.g., positive) phase of a biphasic waveform, while the third waveform segment PW 3 is delivered during a second (e.g., negative) phase of the biphasic waveform. The transition between the positive and negative phases of the biphasic waveform occurs at approximately 5 ms into the duration of the shocking waveform, as indicated by the transition from a positive voltage above 200 V to a negative voltage of slightly less than −200 V. During the negative phase of the biphasic waveform, the shocking waveform experiences a voltage decay from a voltage of slightly less than −200 V to a voltage level of slightly less than −100 V.

The upper panel also illustrates example parallel and series configuration of a capacitor bank that may be utilized to deliver the ascending stepped waveform in accordance with embodiments herein. For example, during the first waveform segment PW1, two 110 μF capacitors may be connected in parallel during the discharge of the first portion of the positive phase of the biphasic waveform. During the third waveform segment PW3, two 110 μF capacitors may be connected in parallel during the discharge of the entire segment of the negative phase of the biphasic waveform. During the second waveform segment PW2 of the positive phase of the biphasic waveform the capacitors are connected in parallel. The first waveform segment PW1 starts at about 400 volts and discharges along a decaying voltage shape (e.g., for about 3 mec.) until reaching about 300V. Thereafter, the capacitor configuration is switch from the series configuration to the parallel configuration and the discharge voltage jumps from about 300V to 600V. The second waveform segment PW2 starts at about 600V, and discharges to along a decaying voltage shape (e.g., for about 3 msec.) until reaching about 320V. At the end of the second waveform segment PW 2, the biphasic waveform switches polarity, as denoted by the transition from the positive voltage (e.g., approximately 320 V when connected in series), to a negative voltage (e.g., approximately 160 V when connected in parallel). At the transition between the second and third waveform segments PW 2 and PW 3, the capacitor configuration switches from the parallel configuration back to the series configuration. The third waveform segment PW3 starts at about 160V, and discharges along a decaying voltage shape until terminating (e.g., after 3-6 msec. and at a voltage of between −75 V and-100 V).

The shape of the decaying voltages during segments PW1-PW3 assumes a shocking load resistance across the shocking electrodes to be approximately 50 ohms, an effective capacitance of 440 μF during segment PW1, an effective capacitance of 110 μF during segment PW2 and an effective capacitance of 440 μF during segment PW3. It is recognized that the starting and ending voltages of each segment and the shape of the decaying voltage over one or more of the segments PW1-PW3 will vary based on variation in the effective capacitance values, the shocking load resistance and the like. It is also recognized that the shocking load resistance will vary between different patient anatomies, electrode combinations/configurations, shocking vectors and the like.

The lower panel illustrates the membrane response experienced by the tissue in connection with each of the waveform segments PW1-PW3 over the duration of the corresponding segments. The membrane response is generated by a cell membrane response model which models or simulates the voltage potential experienced by a cell membrane at a particular point in time when excited by a corresponding voltage potential from the ascending stepped waveform. The lower panel illustrates a normalized membrane response, such as a voltage potential experienced at the member, over time as illustrated along the horizontal axis. During segment PW1, the membrane experiences a response (e.g., voltage potential) that begins at a reference or zero potential and increases along a first response curve over the duration of the first waveform segment PW 1 until reaching a first intermediate voltage potential. The first response curve follows a first non-linear function. The first nonlinear function may represent a first concave curve with a slope that progressively decreases at a first rate, where the change in slope transitions in a positive (e.g., clockwise) direction. At a point of transition from the parallel configuration to the series configuration, the membrane response also transitions to a second response curve. The second response curve follows a second non-linear function. The second nonlinear function may represent a second concave curve with a slope that progressively decreases at a second rate, where the change in slope transitions in a positive (e.g., clockwise) direction. The second response curve is steeper than the first response curve. The second concave response rises from the first intermediate voltage potential over the duration of the second waveform segment PW2 to a second intermediate voltage potential.

At a point of transition from the series configuration back to the parallel configuration, the membrane response also transitions to a third response curve. The third response curve follows a third non-linear function (e.g., a first convex response curve with a slope that progressively changes at a third rate). During delivery of the negative phase of the biphasic waveform, the membrane is restored to a zero potential (e.g., between 9 ms and 10 ms).

Table 3 below illustrates examples of capacitance values that may be utilized by capacitor banks in accordance with embodiments herein. The first row of Table 3 corresponds to a capacitor bank utilized to deliver a standard, conventional biphasic waveform with a 110 μF effective capacitance derived from two 220 μF capacitors in series. Rows 2-6 in Table 3 illustrate combinations of capacitors that may be utilized in capacitor banks in accordance with embodiments herein, with the capacitors being switched between parallel, series, parallel, to derive different shapes for the ascending stepped waveform (e.g., similar to the ascending stepped waveform in the upper panel in FIG. 12B).

TABLE 3

| | | |
|---|---|---|
| Model Designation | Parallel | Series |

| | | | |
|---|---|---|---|
| STD 220 uF x2 | . . . | 110 | uF only |

| | | | | |
|---|---|---|---|---|
| Step 220 uF x2 | 440 | uF | 110 | uF |
| Step 140 uF x2 | 280 | uF | 70 | uF |
| Step 110 uF x2 | 220 | uF | 55 | uF |
| Step 55 uF x2 | 110 | uF | 27.5 | uF |
| Step 27 uF x2 | 54 | uF | 13.5 | uF |

Waveform models were derived, utilizing the capacitor combinations noted in Table 3 (e.g., capacitor banks having two 220 μF capacitors or two 140 μF capacitors or two 110 μF capacitors or two 55 μF capacitors or two 27 μF capacitors), to form ascending stepped waveforms. Ascending stepped waveforms generated by the different capacitor banks were delivered to a cell membrane model. The model generated membrane responses (similar to the lower panel in FIG. 12B). The model was assumed to have a 50 ohm resistance load and a 3.5 msec time constant.

As noted herein, the shocking load resistance across the shocking electrodes may vary between different patients, electrode combinations/configurations, shocking vectors and the like. For example, the load resistance may vary between 20 and 90 ohms, and in some situations be below 20 ohms or above 90 ohms. Accordingly, simulations have been performed to apply the ascending stepped waveform, identified in accordance with new and unique aspects herein, to membrane response models that are assigned various shocking load resistances (e.g., between 20 and 90 ohms).

FIG. 12C illustrates a collection of ascending stepped waveforms and corresponding membrane responses that were modeled utilizing the various shocking load resistances in accordance with embodiments herein. In the upper panel, ascending stepped waveforms are illustrated with the voltage indicated along the vertical axis and time indicated along the horizontal axis. All of the ascending stepped waveforms were generated utilizing two 110 μF capacitors connected in parallel, then the series, then parallel (as described above in connection with FIG. 12B). Each of the ascending stepped waveforms corresponds to a different shocking load resistance, ranging between 20 ohms and 90 ohms with 10 ohm steps therebetween (e.g., 20, 30, 40, 50, etc.). In the example of FIG. 12C, the ascending stepped waveforms 1251-1258 correspond to waveform shapes that would result from shocking load resistances of 20 ohms, 30 ohms, 40 ohms, 50 ohms, 60 ohms, 70 ohms, 80 ohms, and 90 ohms, respectively.

The lower panel of FIG. 12C illustrates membrane responses experienced by cellular membranes, where the vertical axis illustrates a normalized membrane response (e.g., normalized the voltage potential) and the horizontal axis corresponds to the same time axis as used in the upper panel. A collection of membrane responses 1261-1268 are illustrated, where each of the membrane responses 1261-1268 is representative of the voltage potential experienced by a cell membrane when excited by the corresponding ascending stepped waveforms 1251-1258. Further, each of the membrane responses 1261-1268 correspond to different shocking load resistances of 20 ohms, 30 ohms, 40 ohms, 50 ohms, 60 ohms, 70 ohms, 80 ohms, and 90 ohms, respectively.

With respect to the ascending stepped waveform 1251, first and second waveform segments 1248 and 1249 are generated by connecting the combination of 110 μF capacitors in parallel, and then in series, respectively, to form the positive phase of a biphasic waveform. Thereafter, the phase is shifted to generate the negative phase of the biphasic waveform during the third waveform segment 1250. The membrane response 1261 is generated by the cell membrane model in response to the ascending stepped waveform 1251. The membrane response 1261 includes first and second response segments 1268 and 1269, that exhibit increasing voltage potentials, in response to the first and second waveform segments 1248 and 1249, during the positive phase of the biphasic waveform. The membrane response 1261 includes a third response segment 1270, that exhibits a decreasing voltage potential, in response to the third waveform segment 1250.

Each of the additional ascending stepped waveforms 1252-1258 and corresponding membrane responses 1262-1268 exhibit similar behaviors, but with different time constants, due to the differences in shocking load resistance. Each of the membrane responses 1261-1268 reach a common desired (e.g., peak) membrane voltage potential (normalized to a value of 1). It is understood that the discussion hereafter in connection with a “peak membrane response” or “peak membrane voltage potential” refers to a local or desired maximum, and not a physiologic maximum. The membrane may be capable of additional response and may be capable of experiencing a higher voltage potential. Instead, the peak, as used herein, may be arbitrarily chosen as an amount of response and/or voltage potential, sufficient to achieve a desired physiologic effect (e.g., defibrillation). As a further clarification, the peak membrane response does not necessarily correspond to a defibrillation threshold of an individual patient, but instead is used as a reference level for comparison between shocking waveforms having different shapes, energies and the like.

To achieve a common peak membrane voltage potential, the initial charge voltage on the corresponding capacitor bank was adjusted (as illustrated along the vertical axis in the upper panel. By way of nonlimiting examples, when the shocking load resistance was 20 ohms, the initial charge voltage on the capacitor bank was increased above 500 V (e.g., do a proximally 600 V), as indicated by the starting voltage of the ascending stepped waveform 1251. As another nonlimiting example, when the shocking load resistance was assumed to be 40 ohms, the initial charge voltage in the capacitor bank was set to approximately 500 V, as indicated by the starting voltage of the ascending stepped waveform 1252. As the shocking load resistance increases at each step above 40 ohms, the initial charge voltage of the corresponding ascending stepped waveform 1253-1258 is decreased by a corresponding amount sufficient to ensure that the ascending stepped waveform achieved a peak membrane response of a desired voltage potential (normalized to 1 in the lower panel).

The duration of the third waveform segment, corresponding to the negative phase of the biphasic waveform, was selected to be sufficiently long to enable the membrane response to return to a membrane voltage potential of approximately zero. By way of example, with respect to the ascending stepped waveform 1251, the duration of the third waveform segment extends from a time point in the duration of the shocking waveform at approximately 3.6 ms until a point in time in the duration of the shocking waveform at approximately 9.6 ms. As another nonlimiting example, with respect to the ascending stepped waveform 1258, the third waveform segment may extend from a time point in the duration of the shocking waveform at approximately 6.4 ms to a time point in the duration of the shocking waveform at approximately 10.4 ms.

FIG. 12D illustrates predicted delivered energy as a function of the shocking load resistances in accordance with embodiments herein. The vertical axis illustrates an approximation of the total energy delivered during a biphasic waveform, while the horizontal axis illustrates the shocking load resistance. The shocking load resistances were described in connection with FIG. 12C and the effective capacitances are described in connection with Table 3. The graph 1271 plots combinations of delivered energy and shocking load resistance in connection with utilizing a standard biphasic waveform delivered from a capacitor bank connected in series throughout delivery of the shock. The remaining graphs plot combinations of delivered energy and shocking load resistance in connection with utilizing a capacitor bank switched between parallel, series, parallel configurations to form an ascending stepped biphasic waveform. By way of example, the graph 1272 corresponds to a capacitor bank utilizing two 27 μF capacitors switched between parallel, series, parallel configurations, while the graphs 1273-1276 correspond to capacitor banks, each of which utilizes two capacitors (55 μF capacitors, two 110 μF capacitors, two 140 μF capacitors, and two 220 μF capacitors, respectively).

The predicted energy deliveries in FIG. 12D represent the amount of energy needed to achieve the peak membrane response (e.g., the normalized value of 1 in the membrane responses illustrated in the lower panel of FIG. 12C). For example, with reference to FIGS. 12C and 12D, the ascending stepped waveform utilizing the 110 μF capacitor bank (e.g., two 110 μF capacitors switched between parallel, series in parallel) was modeled to need to deliver approximately 10 J into a shocking load resistance of 30 ohms in order to achieve the peak membrane response (corresponding to the peak of the membrane response 1264 in FIG. 12C). As another example, the 110 μF capacitor bank was modeled to need to deliver approximately 15 J into a 30 ohm load resistance in order to achieve the corresponding peak membrane response (e.g., the peak of response 1263 in FIG. 12C).

For a standard biphasic waveform using series connected 110 μF capacitors, approximately 30 J (as denoted at 1278) is necessary to achieve a peak membrane response when the shocking load resistance is approximately 20 ohms. Alternatively, when the ascending stepped waveform is utilized with capacitance banks having two capacitors with capacitances of between 110 μF and 220 μF, the delivered energy is modeled to achieve peak membrane response with a delivered energy of between 21 J and 24 J (as denoted at 1277) when the shocking load resistances approximately 20 ohms. As another example, with reference to the data points at 1279, when the shocking load resistance is approximately 50 ohms, to achieve the peak membrane response, the standard biphasic waveform needs to deliver approximately 11.2 J. In contrast, the modeled collection of ascending stepped waveforms were able to achieve the peak membrane response while delivering only between 8-12 J. As a further example, the 220 μF configuration (e.g., utilizing two 220 μF capacitors to form the ascending stepped waveform) only needed to deliver approximately 10 J of energy (see the data point at 1280) with the 50 ohm shocking load resistance in order to achieve the peak membrane response.

The foregoing models predict that the ascending stepped waveforms reduce the energy that is necessary to achieve peak membrane response. For example, the model predicts that the ascending stepped waveform associated with the 220 μF configuration (e.g., two 220 μF capacitors switched between parallel, series, parallel configurations) would reduce the delivered energy, needed to achieve peak membrane response, by approximately 21%, as compared to the energy that must be delivered by the conventional standard 110 μF series waveform to achieve peak membrane response. In fact, each of the ascending stepped waveform configurations achieve energy reduction over the standard waveform at substantially all load resistances, but for the 27 μF configuration. In the 27 μF configuration, the energy needed to achieve peak membrane response is higher than, or the same as, the energy demand of the conventional standard waveform for 20-40 ohm load resistances.

FIG. 12E illustrates predicted peak voltage as a function of the shocking load resistances in accordance with embodiments herein. The vertical axis illustrates an approximation of the peak voltage delivered during a biphasic waveform, while the horizontal axis illustrates the shocking load resistance. The shocking load resistances are described in connection with FIG. 12C and the effective capacitances are described in connection with Table 3. The graph 1281 plots combinations of peak voltage and shocking load resistance in connection with a standard biphasic waveform delivered from a capacitor bank connected in series throughout delivery of the shock. The remaining graphs plot combinations of peak voltage and shocking load resistance in connection with utilizing a capacitor bank switched between parallel, series, parallel configurations to form an ascending stepped biphasic waveform. By way of example, the graph 1282 corresponds to a capacitor bank utilizing two 27 μF capacitors switched between parallel, series, parallel configurations, while the graphs 1283-1286 correspond to capacitor banks, each of which utilizes two capacitors (55 μF capacitors, two 110 μF capacitors, two 140 μF capacitors, and two 220 μF capacitors, respectively).

For a standard biphasic waveform using series connected 110 μF capacitors, over 700 V (as denoted at 1288) is necessary as the peak voltage to achieve a peak membrane response when the shocking load resistance is approximately 20 ohms. Alternatively, when the ascending stepped waveform is utilized with switched capacitance banks having capacitors with capacitances of between 110 μF and 220 μF, the model predicts that the peak membrane response can be achieved while delivering a peak voltage of between 350 V and 675 V (at 1289) when the shocking load resistance is approximately 20 ohms. As another example, with reference to the data points at 1290, when the shocking load resistance is approximately 50 ohms, to achieve the peak membrane response, the standard biphasic waveform needs to utilize a peak voltage of greater than 450 V. In contrast, the modeled collection of ascending stepped waveforms were able to achieve the peak membrane response while delivering peak voltages of 375-450 V. As a further example, the 220 μF configuration (e.g., utilizing two 220 μF capacitors to form the ascending stepped waveform) was able to utilize a peak voltage of 21% less than the peak voltage utilized by the conventional biphasic waveform with the 50 ohm shocking load resistance in order to achieve the peak membrane response. The ascending stepped waveform configurations that utilized 110 μF, 140 μF and 220 μF capacitors all utilized lower peak voltages as compared to the conventional biphasic waveform.

The foregoing models predict that the ascending stepped waveforms reduce the peak voltage that is necessary to achieve peak membrane response. For example, the model predicts that the ascending stepped waveform associated with the 220 μF configuration (e.g., two 220 μF capacitors switched between parallel, series, parallel configurations) would reduce the peak voltage, needed to achieve peak membrane response, by approximately 21%, as compared to the peak voltage that must be utilized by the conventional standard 120 μF series waveform to achieve peak membrane response.

FIG. 12F illustrates the energy predictions of FIG. 12D normalized as a function of shocking load resistance. The delivered energy for each respective load resistance data point, associated with the ascending stepped waveform, was divided by the delivered energy of the standard biphasic waveform at the corresponding shocking load resistance. From FIG. 12F, it can be seen that, for load resistances above 40 ohms, the energy utilized by the ascending stepped waveforms is approximately 80% of the delivered energy of the conventional biphasic waveform, ignoring the delivered energies associated with the 27 μF capacitor configuration.

FIG. 12G illustrates the voltage predictions of FIG. 12E normalized as a function of shocking load resistance. The peak voltage for each respective load resistance, associated with the ascending stepped waveform, was divided by the peak voltage of the standard biphasic waveform at the corresponding shocking load resistance. From FIG. 12G, it can be seen that, the model predicts lower peak voltages (approximately 21% lower) for the 110 μF, 140 μF and 220 μF capacitor configurations used with the ascending stepped waveform as compared to the peak voltage for the standard biphasic waveform.

In accordance with new and unique aspects herein, the negative phase of the ascending stepped waveform is generated utilizing a parallel connection between the capacitors the capacitor bank. In certain implementations, it is preferable to use the parallel connection between the capacitors during the negative phase of the ascending stepped waveform, such as when it is desirable to limit variation in the duration of the negative phase (corresponding to the third pulse width PW 3 discussed above). The parallel capacitor connection exhibits a behavior that is more independent of the shocking load resistance. More specifically, when the negative phase of the ascending stepped waveform utilize the two 220 μF capacitors connected in parallel, the voltage potential across the membrane was able to be reduced to zero with negative phases having durations of between four and 5 ms, when considering all shocking load resistances (e.g., between 20 and 90 ohms). In contrast, when the negative phase of the ascending stepped waveform delivered utilizing capacitors connected in series, the duration of the negative phase ranged from 5.5 ms for a 20 ohm load resistance down to 2.8 ms for a 90 ohm load resistance. Accordingly, the duration of the negative phase exhibited a substantially larger variation at different loads resistances when two 220 μF capacitors are connected in series.

Alternatively, in other instances, it may be desirable to connect the capacitors in series during the negative phase (PW3) of the ascending stepped waveform. For example, a serious connection of the capacitors may be used when the variation in the duration of the negative phase (e.g., PW3) is of less concern, or when the shocking load resistance is known to have a level, or be within a range, for which the corresponding duration of the negative phase series connected capacitors is acceptable.

In accordance with the discussion above, new and unique aspects herein implement an ascending stepped waveform that is generated through switching connections of capacitors between parallel, serial, parallel configurations to achieve a reduction in energy and peak voltage, as compared to conventional waveforms. As noted herein, the delivered energy and peak voltage reductions may be approximately 21% relative to delivered energy and peak voltage of a conventional biphasic waveform. As further noted herein, capacitor banks that utilize two 110 μF, 140 μF and 220 μF capacitors exhibit similar reductions in delivered energy and peak voltage, relative to a conventional biphasic waveform. Accordingly, it may be preferable to utilize capacitor banks having pairs of capacitors, each of which has capacitances of between 110 and 220 μF. Additionally, it may be more preferable to utilize the capacitor bank having a pair of capacitors, each of which has a capacitance of between 110 and 140 μF, and even more preferably to utilize a capacitor bank having a pair of capacitors, each of which has a capacitance of 110 μF.

In accordance with new and unique aspects herein, the ascending stepped waveform enables an implantable medical device to be reduced in size by avoiding the need for a truncated exponential biphasic waveform that uses two 220 μF capacitors connected in series. The reduction in size is achieved, in part, by utilizing physically smaller capacitors, each of which has a capacitance of 110 μF.

In accordance with new and unique aspects herein, the ascending stepped waveform utilizes less energy from the battery of the implantable medical device, as compared to a conventional truncated biphasic waveform. By using two 110 μF capacitors, the implantable medical device is able to reform the capacitors with approximately half the charge from the battery, that would otherwise be necessary for reforming two 220 μF capacitors connected in series to generate a conventional truncated exponential biphasic waveform. By reducing the charge necessary to reform the capacitors, the longevity of the battery is increased (e.g., by 10% or more).

Next, the discussion turns to managing the pulse width of each of the waveform segments in the ascending stepped waveform. The pulse width of the waveform segments may vary depending upon various factors, such as the shocking load resistance, capacitance of the capacitor is utilized in the capacitor bank and the like.

FIG. 12H illustrates graphs plotting examples of pulse widths that may be utilized in connection with the first, second and third waveform segments (denoted phase 1, 2, 3) of the ascending stepped waveform to achieve a desired membrane response. The graph corresponds to the capacitor bank that utilizes a pair of capacitors, each of which has a capacitance of 110 μF. As noted in FIG. 12H, the pulse width of the first waveform segment (denoted “phase 1” in FIG. 12H and corresponding to PW1 in FIG. 12B) corresponds to the first portion of the first (e.g., positive) phase of the biphasic waveform and ranges between 2.5 ms and 3.4 ms for load resistances of 20-90 ohms. The pulse width of the second waveform segment (denoted “phase 2” in FIG. 12H and corresponding to PW2 in FIG. 12B) corresponds to the second portion of the first phase of the biphasic waveform and ranges between 1 ms and 3 ms for load resistances between 20-90 ohms. The pulse width of the third waveform segment (denoted “phase 3” in FIG. 12H and corresponding to PW3 in FIG. 12B) corresponds to the second (e.g., negative) phase of the biphasic waveform and ranges between 6.2 ms and 4.4 ms for load resistances between 20-90 ohms.

FIG. 12I illustrates graphs plotting examples of pulse widths that may be utilized in connection with the first, second and third waveform segments (denoted phase 1, 2, 3) of the ascending stepped waveform to achieve a desired membrane response. The graph corresponds to the capacitor bank that utilizes a pair of capacitors, each of which has a capacitance of 220 μF. As noted in FIG. 12I, the pulse width of the first waveform segment (denoted “phase 1”) corresponds to the first portion of the first (e.g., positive) phase of the biphasic waveform and ranges between 3 ms and 3.6 ms for load resistances of 20-90 ohms. The pulse width of the second waveform segment (denoted “phase 2”) corresponds to the second portion of the first phase of the biphasic waveform and ranges between 1.7 ms and 4.3 ms for load resistances between 20-90 ohms. The pulse width of the third waveform segment (denoted “phase 3”) corresponds to the second (e.g., negative) phase of the biphasic waveform and ranges between 4.8 ms and 4.2 ms for load resistances between 20-90 ohms.

FIG. 12J illustrates graphs plotting examples of pulse widths that may be utilized in connection with the first, second and third waveform segments (denoted phase 1, 2, 3) of the ascending stepped waveform to achieve a desired membrane response. The graph corresponds to the capacitor bank that utilizes a pair of capacitors, each of which has a capacitance of 220 μF. As noted in FIG. 12J, the pulse width of the first waveform segment (denoted “phase 1”) ranges between 3 ms and 3.6 ms for load resistances of 20-90 ohms. The pulse width of the second waveform segment (denoted “phase 2”) corresponds to the second portion of the first phase of the biphasic waveform and ranges between 1.7 ms and 4.3 ms for load resistances between 20-90 ohms. Two examples are illustrated for the third waveform segment, namely a phase 3 in which the capacitors are connected in parallel and a phase 3 in which the capacitors are connected in series. The pulse width of the parallel third waveform segment (denoted “phase 3 parallel”) corresponds to the second (e.g., negative) phase of the biphasic waveform and ranges between 4.8 ms and 4.2 ms for load resistances between 20-90 ohms. The pulse width of the series third waveform segment (denoted “phase 3 series”) ranges between 5.5 ms and 2.8 ms for load resistances between 20-90 ohms.

FIG. 12K illustrates graphs plotting examples of pulse widths that may be utilized in connection with the first and second phases of a conventional truncated exponential biphasic waveform the conventional waveform includes a first (e.g., positive) phase and a second (e.g., negative) phase. The conventional waveform follows an exponentially decaying curve over the first phase and an exponentially decaying curve over the second phase. The graph 12K corresponds to the conventional capacitor bank that connects in series a pair of capacitors, each of which has a capacitance of 220 μF. As noted in FIG. 12K, the pulse width of the first phase (denoted “phase 1”) ranges between 2.7 ms and 5.7 ms for load resistances of 20-90 ohms. The pulse width of the second waveform segment (denoted “phase 2”) ranges between 5.3 and 2.7 ms for load resistances between 20-90 ohms.

The graphs in FIGS. 12H-12K illustrate that the duration of the first, second and third waveform segments exhibit less variation, when exposed to different load resistances, as compared to the pulse duration exhibited by the first and second phases of the conventional exponentially decaying biphasic waveform.

FIG. 12L illustrates a series of theoretical responses for membrane potentials that are calculated from convolutions of corresponding defibrillation forcing functions and a predetermined consistent membrane impulse response. The defibrillation forcing functions are shaped differently from one another based on the effective capacitance of the capacitor bank utilized to form/deliver the defibrillation forcing function. In the example of FIG. 12L, the defibrillation forcing functions (and corresponding membrane potentials) correspond to a range of effective capacitances between 110 μF and 140 μF.

In accordance with new and unique aspects herein, an implantable medical device is provided that comprises electrodes configured to sense cardiac events; a charging circuit; a reconfigurable capacitor bank that includes capacitors; a switching circuit coupled between the reconfigurable capacitor bank and the output; and a parallel/series reconfiguration (PSR) circuit that interconnects the capacitors in parallel and series configurations, the PSR circuit configured to switch between the parallel and series configurations during delivery of a shock to define an ascending stepped waveform. The shape of the ascending stepped waveform includes a positive phase segment that includes first and second waveform segments that are defined by the parallel and series connections, respectively, between the capacitors. The ascending stepped waveform may represent a biphasic waveform, wherein the PSR circuit is configured to connect the capacitors in the parallel configuration during the discharge of a first portion of a positive phase of the biphasic waveform. The PSR circuit is further configured to switch the capacitors from the parallel configuration to the series configuration during the discharge of a second portion of the positive phase of the biphasic waveform. In accordance with new and unique aspects herein, additionally or alternatively, the PSR circuit may be configured to switch the capacitors from the series configuration back to the parallel configuration during the discharge of a negative phase of the biphasic waveform. Additionally or alternatively, the PSR circuit may be configured to switch the capacitors from the parallel configuration, to the series configuration and back to the parallel configuration at intermediate points during discharge of the shock. In accordance with new and unique aspects herein, as explained above, the reconfigurable capacitor bank includes first and second capacitors, each of which has an individual capacitance value of between 55 μF and 220 μF. The PSR circuit switch an effective capacitance of the reconfigurable capacitor bank between 27.5 μF and 440 μF when switching between the series configuration and the parallel configuration, respectively.

While the foregoing discussion describes the formation of the ascending stepped waveform in connection with switching a capacitor bank between parallel and series configurations, it is recognized that various combinations of the embodiments herein may be utilized in combination. For example, the output control circuit of FIG. 6 may be utilized in combination with a reconfigurable capacitor bank to further define the shapes of the first, second and/or third waveform segments of the ascending stepped waveform. For example, the output control circuit may be configured to generate a control signal that includes control pulses that control the switching circuit to switchably connect the configurable capacitor bank to the electrodes. The output control circuit may vary a duty cycle of the control pulses in a manner that defines a shape of the first, second and/or third waveform segments for one or more shocks.

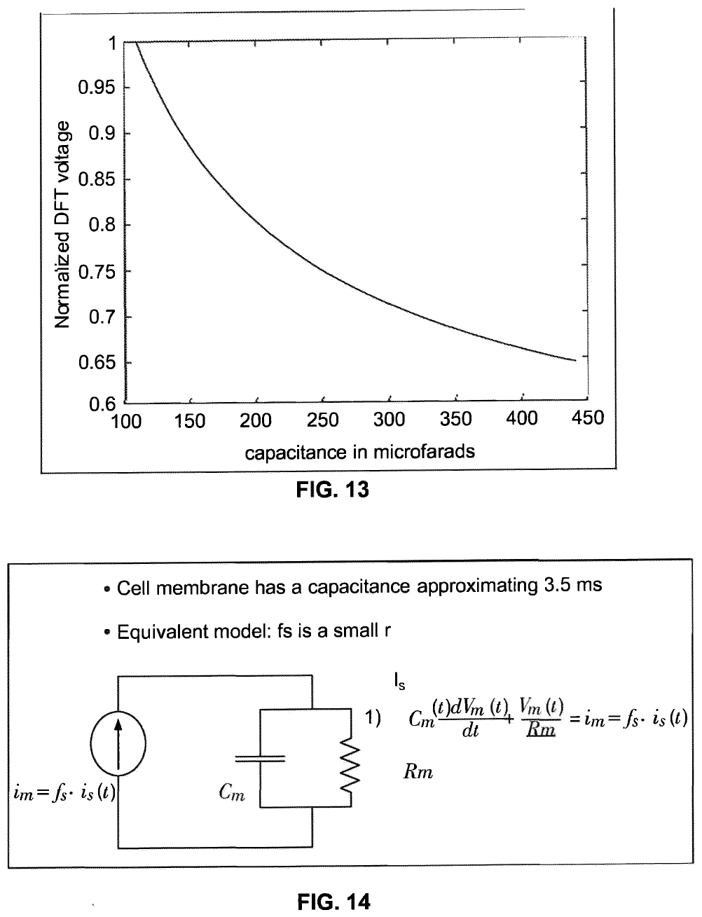

FIG. 13 illustrates a graph plotting different effective capacitances (along the horizontal axis) and normalized DFT voltages (along the vertical axis). As shown in FIG. 13, effective capacitances may range between 110 μF and 440 μF, while the corresponding normalized DFT voltage ranges from one down to 0.65. From the graph of FIG. 13, it can be seen that the DFT voltage may be lowered as the effective capacitances increased, although a diminishing return is exhibited in that the incremental additional increase in DFT reduction continuously decreases as the effective capacitance increases above 440 μF.

Next, the discussion turns to an electrical model that may be utilized to model the membrane response when exposed to various defibrillation shocks, such as the conventional exponentially decaying biphasic waveform and the various ascending stepped waveforms described in connection with FIGS. 12A-12L. For example, with respect to FIG. 12A, the exponentially decaying biphasic waveform (upper left panel) may be applied to the equations of FIGS. 14-18 to predict the membrane response illustrated in the upper right panel. The equations also may be used to predict the membrane response in the lower right panel when a defibrillation shock is applied having the exponentially decaying biphasic waveform in the lower left panel. With respect to FIG. 12B, the ascending stepped waveform shown in the upper panel is applied to the equations of Figures of 14-18 to predict the membrane response shown in the lower panel. With respect to FIG. 12C, the collection of ascending stepped waveforms shown in the upper panel or separately applied to the equations of FIGS. 14-18 to predict the collection of membrane response as shown in the lower panel. The equations 14-18 are similarly used to obtain the energy, voltage and waveform segment durations data points in FIGS. 12D-12L.

FIG. 14 illustrates an electrical model for a myocardial cellular membrane. The model of FIG. 14 further illustrates why the larger effective capacitance of yields a better DFT result when delivered with a common voltage. In the model of FIG. 14, the cellular membrane is modeled

$$\\ \begin{matrix}{C}_{m} \frac{( t ) {dV}_{m} ( t )}{dt} + \frac{{V}_{m} ( t )}{Rm} = {i}_{m} = {f}_{s} \cdot {i}_{s} ( t ) , & 1 )\end{matrix}$$

to have a capacitance that yields a time constant tₘ of approximately 3.5 ms.

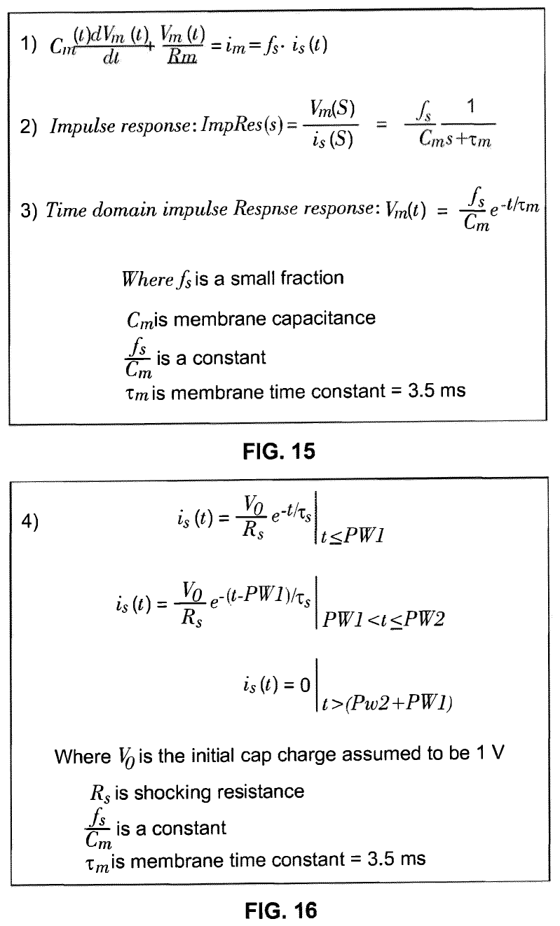

FIG. 15 illustrates a series of equations to model the membrane impulse response in accordance with embodiments herein.

FIG. 16 illustrates a series of equations to model the defibrillation forcing function in accordance with embodiments herein.

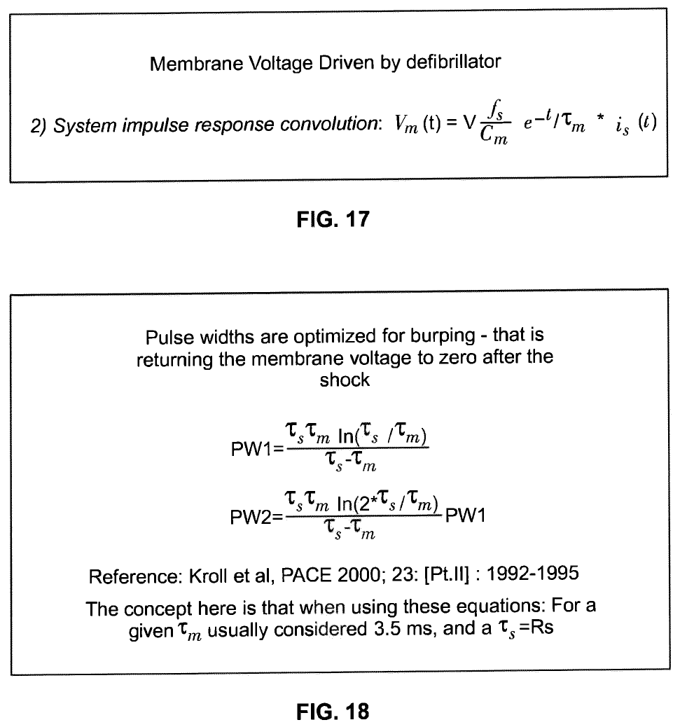

FIG. 17 illustrates an equation to model the membrane voltage/potential experienced by the myocardial tissue when driven by a defibrillation shock in accordance with embodiments herein.

FIG. 18 illustrates equations to calculate pulse widths for the pulses of one or more of the shocks in the phase I therapy, the pulse train in the phase II therapy, and/or the pacing pulses in the phase III therapy. The pulse widths may be defined (e.g., optimized) in order to achieve “burping”, namely, to return the membrane potential to zero following a corresponding shock. The pulse widths may be defined in accordance with the process described in the Kroll et al. publication incorporated herein by reference in its entirety.

In accordance with embodiments herein, increasing the effective capacitance that is utilized in connection with delivering one or more MV shocks will reduce the voltage requirement for the initial charge of the capacitor bank. For example, utilizing an effective capacitance of approximate 440 μF can reduce the voltage requirement by approximate 35%. As another example, delivering an MV shock having a waveform defined by an effective capacitance of approximately 440 μF and an initial maximum voltage of 490 V is substantially equivalent to delivering an HV shock having a waveform defined by an effective capacitance of approximately 110 μF and an initial maximum voltage of 753 V.

The model described in connection with FIGS. 14-18 is defined in terms of a current power source. However, it is recognized that a substantially similar model may be described in terms of a voltage power source. For example, the model may be defined based on the following equations.

$$\\ \begin{matrix}Nodal Equation : {C}_{m} \frac{{dV}_{m} ( t )}{dt} + \frac{{V}_{m} ( t )}{Rm} = {i}_{m} = {f}_{s} \cdot {V}_{s} ( t ) & 1 )\end{matrix}$$

$$\\ \begin{matrix}Impulse Response response : {V}_{m \_ imp} ( t ) = \frac{{f}_{s}}{{C}_{m}} {e}^{{}_{} - t / {τ}_{m}} & 2 )\end{matrix}$$

$$\\ \begin{matrix}{τ}_{m} = Rm \cdot {C}_{m} & 3 )\end{matrix}$$

$$\\ \begin{matrix}Convolution : {V}_{m} ( t ) = \frac{{f}_{s}}{{C}_{m}} {\int }_{- ∞}^{∞} {V}_{s} ( τ - t ) {e}^{{}_{} - τ / {τ}_{m}} dt & 4 )\end{matrix}$$

$$\\ \begin{matrix}Delivered Energy : {E}_{d} ( t ) = \frac{{f}_{s}}{{C}_{m}} \frac{1}{{R}_{s}} {\int }_{- ∞}^{∞} {V}_{s}^{{}_{} 2} ( t ) dt & 5 )\end{matrix}$$

In the above equations, the value fₛ represents a small fraction, while Cₘ represents the membrane capacitance, fₛ/Cₘ is a constant and tₘ is the membrane time constant (e.g., 3.5 ms). The convolution in equation 4) may be rewritten as follows, where V₀ is the initial capacitor charge assumed to be a normalized voltage (1 V), Rₛ is the shocking load resistance, fₛ/Cₘ is a constant and tₘ is the membrane time constant:

$${\\ {V}_{s} ( t ) = \frac{{V}_{0}}{{R}_{s}} {e}^{{}_{} - t / {τ}_{s}} \vert}_{t \leq PW 1}$$

$${\\ {V}_{s} ( t ) = \frac{{V}_{0}}{{R}_{s}} {e}^{{}_{} - ( t - PW 1 ) / {τ}_{s}} \vert}_{PW 1 < t \leq Pw 2}$$

$${\\ {V}_{s} ( t ) = 0 \vert}_{t > ( Pw 2 + PW 1 )}$$

The system impulse response convolution can then be calculated as follows:

$$\\ \begin{matrix}System impulse response convolution : {V}_{m} ( t ) = V \frac{{f}_{s}}{{C}_{m}} {e}^{{}_{} - t / {τ}_{m}} * {i}_{s} ( t ) & 2 )\end{matrix}$$

The following equations may be utilized to calculate the durations for the pulse width sufficient to achieve “burping”, namely, to return the membrane voltage to 0 following a shock.

$$\\ PW 1 = \frac{{τ}_{s} {τ}_{m} ln ( {τ}_{s} / {τ}_{m} )}{{τ}_{s} - {τ}_{m}}$$

$$\\ PW 2 = \frac{{τ}_{s} {τ}_{m} ln ( 2 * {τ}_{s} / {τ}_{m} )}{{τ}_{s} - {τ}_{m}} - PW 1$$

Closing Statements

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable medical device, comprising:
electrodes configured to sense cardiac events;
a charging circuit;
a reconfigurable capacitor bank that includes capacitors;
a switching circuit coupled between the reconfigurable capacitor bank and the output; and
a parallel/series reconfiguration (PSR) circuit that interconnects the capacitors in parallel and series configurations, the PSR circuit configured to switch between the parallel and series configurations during delivery of a shock to define an ascending stepped waveform.

2. The device of claim 1, wherein the shape of the ascending stepped waveform includes a positive phase segment that includes first and second waveform segments that are defined by the parallel and series configurations, respectively, of the capacitors.

3. The device of claim 1, wherein the ascending stepped waveform represents a biphasic waveform, the PSR circuit configured to connect the capacitors in the parallel configuration during the discharge of a first portion of a positive phase of the biphasic waveform, the PSR circuit configured to switch the capacitors from the parallel configuration to the series configuration during the discharge of a second portion of the positive phase of the biphasic waveform.

4. The device of claim 3, wherein the PSR circuit is configured to switch the capacitors from the series configuration back to the parallel configuration during the discharge of a negative phase of the biphasic waveform.

5. The device of claim 1, wherein the PSR circuit is configured to switch the capacitors from the parallel configuration, to the series configuration and back to the parallel configuration at intermediate points during discharge of the shock.

6. The device of claim 1, wherein the reconfigurable capacitor bank includes first and second capacitors, each of which has an individual capacitance value of between 55 μF and 220 μF, the PSR circuit switching an effective capacitance of the reconfigurable capacitor bank between 27.5 μF and 440 μF when switching between the series configuration and the parallel configuration, respectively.

7. The device of claim 1, further comprising an output control circuit configured to generate a control signal that includes control pulses that control the switching circuit to switchably connect the configurable capacitor bank to the electrodes, the output control circuit configured to vary a duty cycle of the control pulses in a manner that defines a shape of one or more of first, second or third waveform segments of the shock.

8. The device of claim 1, further comprising an output control circuit configured to generate a control signal that includes control pulses that control the switching circuit to switchably connect the configurable capacitor bank to the output, the output control circuit configured to vary a duty cycle of the control pulses between at least first and second duty cycles during a positive phase segment of a stimulation waveform, based at least in part on a shocking load resistance across shocking electrodes and a membrane response model of the tissue, in a manner that defines a shape of the positive phase segment of the stimulation waveform for one or more shocks, wherein the output control circuit is configured to vary the first and second duty cycles to define the positive phase segment of the stimulation waveform to have a leading edge profile having an ascending non-linear shape that transitions from a low voltage to a high voltage, and a steady-state plateau at the high voltage for a duration of the positive phase segment.

9. The device of claim 8, wherein the positive phase segment is defined by a collection of the control pulses, in which the duty cycle varies such that an initial portion of the collection includes a first duty cycle and a final portion of the collection includes a second duty cycle that is longer than the first duty cycle, wherein the first and second duty cycles are based in part on the shocking load resistance.

10. The device of claim 8, wherein the output control circuit is configured to increase the duty cycle of successive control pulses, within the control pulses, from an initial shorter duty cycle to a final longer duty cycle based at least in part on the shocking load resistance across shocking electrodes and the membrane response model of the tissue, the first and second duty cycles comprising the initial shorter duty cycle and the final longer duty cycle, respectively.

11. A method, comprising:
sensing cardiac events of a heart;
utilizing one or more processors to declare a ventricular fibrillation (VF) episode based on the cardiac events and initiate charge of a reconfigurable capacitor bank based on the declaration of the VF episode;
switchably connect capacitors of the reconfigurable capacitor bank to the output to deliver a shock; and
during delivery of the shock, connecting the capacitors in a parallel configuration and switching from the parallel configuration to a series configuration to define an ascending stepped waveform for the shock.

12. The method of claim 11, wherein the shape of the ascending stepped waveform includes a positive phase segment that includes first and second waveform segments that are defined by the parallel and series configurations, respectively, of the capacitors.

13. The method of claim 11, wherein the ascending stepped waveform represents a biphasic waveform, the connecting further including connecting the capacitors in the parallel configuration during the discharge of a first portion of a positive phase of the biphasic waveform, and switching the capacitors from the parallel configuration to the series configuration during the discharge of a second portion of the positive phase of the biphasic waveform.

14. The method of claim 13, further comprising switching the capacitors from the series configuration back to the parallel configuration during the discharge of a negative phase of the biphasic waveform.

15. The method of claim 11, wherein the connecting comprises switching the capacitors from the parallel configuration, to the series configuration and back to the parallel configuration at intermediate points during the discharge of the shock.

16. The method of claim 11, further comprising generating a control signal that includes control pulses that control a switching circuit to switchably connect the configurable capacitor bank to the electrodes, the circuit signal varying a duty cycle of the control pulses in a manner that defines a shape of one or more of first, second or third waveform segments of the shock.

17. The method of claim 11, further comprising:
switchably connect the capacitors to the output based on a control signal;
generate the control signal to include control pulses having a duty cycle;
varying the duty cycle of the control pulses between at least first and second duty cycles during a positive phase segment of a stimulation waveform, based at least in part on a shocking load resistance across shocking electrodes and a membrane response model of the tissue, in a manner that defines a shape of the positive phase segment of the stimulation waveform for one or more shocks, wherein the first and second duty cycles are further varied to define the positive phase segment of the stimulation waveform to have a leading edge profile having an ascending non-linear shape that transitions from a low voltage to a high voltage, and a steady-state plateau at the high voltage for a duration of the positive phase segment.

18. The method of claim 17, further comprising utilizing a collection of the control pulses to define the positive phase segment, in which the duty cycle varies such that an initial portion of the collection includes a first duty cycle and a final portion of the collection includes a second duty cycle that is longer than the first duty cycle, wherein the first and second duty cycles are based in part on the shocking load resistance.

19. The method of claim 17, further comprising storing a full charge on the capacitor with a voltage of at least 400V, and varying the duty cycle of the control pulses to define the stimulation waveform of a first shock to have a voltage of less than 300V.
